(19) United States
(12) Patent Application Publication
(10) Pub. No.: US 2026/0273203 A1
(43) Pub. Date: Sep. 17, 2026

(54) ARTICLE FOR DRY POWDER INHALER WITH INDICIA ON UPSTREAM END

(71) Applicant: PHILIP MORRIS PRODUCTS S.A., Neuchâtel (CH)

(72) Inventor: Antonio CONSOLANTE, Casalecchio di Reno (IT)

(21) Appl. No.: 18/869,028

(22) PCT Filed: May 26, 2023
(86) PCT No.: PCT/IB2023/055427

§ 371 Date: Nov. 25, 2024

(30) Foreign Application Priority Data
May 27, 2022 (EP) 22175832.9

(51) Int. Cl. A61M 15/00

(52) U.S. Cl. CPC A61M 15/003; A61M 15/009; A61M 2202/064

(57) ABSTRACT

Disclosed herein is an inhaler article (100) for insertion into a holder to form an inhaler system for providing a compound to lungs of a user, the inhaler article comprising a tubular body extending along a longitudinal axis from a downstream mouthpiece end (153) to an upstream end (152); the upstream end comprising an upstream element; the upstream element comprising an upstream end surface; and wherein the upstream end surface comprises colored indicia (155).

The present disclosure relates to an inhaler article for insertion into a holder to form an inhaler system for delivering an aerosolized active ingredient to the lungs of a user, where the inhaler article is a tubular body extending along a longitudinal axis from a downstream mouthpiece end to an upstream end, each of the downstream mouthpiece end and the upstream end have an end face, and wherein the upstream end face has indicia.

Inhaler systems may have two parts, an inhaler article and a holder. The inhaler article may contain an active ingredient. When combined, the inhaler article and the holder form an inhaler system. The inhaler article has an upstream end, which is inserted into the holder for use, and a downstream or mouthpiece end. In use, the downstream end is contacted by the mouth of a user. When the inhaler article is inserted into the holder, the inhaler article is activated. The inhaler article can be activated by, for example, piercing a capsule inside the inhaler article to release pharmaceutically active powder contained in the capsule. Or, the inhaler article can be activate by, for example, heating an aerosol-generating substrate contained in the inhaler article. Or, the inhaler article can be activated by, for example, the initiation of an airflow through the inhaler system. The user “puffs” on the downstream end of the activated inhaler article. The user's puff initiates an airflow through the inhaler article which delivers an aerosolized active ingredient to the lungs of a user. Or, the inhaler article may be activated by a combination of actions. For example, the user's puff may trigger a heater.

Traditional smoking articles, such as cigarettes, have an upstream end (the lit-end) and a downstream end (the mouth-end). In traditional smoking articles, it is easy to distinguish the upstream end from the downstream end, because tobacco is traditionally visible at the upstream end, or the lit-end, while the downstream end traditionally has a filter. In traditional smoking articles, before the inhaler article is used, the user can see tobacco contained inside a wrapper when the user looks at the upstream end face, the lit-end, of the traditional smoking article. In a traditional smoking article, before it is used, the user can see the end face of a filter wrapped in a paper wrapper when the user looks at the downstream end face, the mouth-end of the traditional smoking article. The end face of the filter at the downstream end may show an end face of a filter, and may appear white, for example. A traditional smoking article is consumed during use. The upstream end (the lit-end) is consumed or burned so that the upstream end of the traditional smoking article is no longer present, and no longer visible, at the end of the smoking experience.

In traditional smoking articles, there may be colored indicia on the wrapper on the outside circumference of a traditional smoking article at the downstream or filter end. For example, the wrapper of a traditional cigarette may be colored at the filter end. This color may be an indicia of origin of the product, the strength of the product, flavor, or other information. This indicia is helpful to the consumer to identify the product. Traditionally, indicia is on the outside of the article, not on either the upstream end face or on the downstream end face of the article.

Traditional smoking articles do not have an element on the upstream end of the smoking article. The upstream end of a traditional smoking article is the lit end. The upstream end in traditionally smoking article is set afire, and aerosol released from the firing of aerosol-generating substrates are inhaled. Any element at the upstream end of a traditional smoking article would be set afire, and aerosol released from burning such an element at the upstream end would be inhaled.

In the dry powder inhaler article disclosed herein, the upstream end is not lit in use. Therefore, the upstream end of the inhaler article can take on utility that is not desired in traditional smoking devices.

Inhaler articles that are structured and arranged for engagement with a holder to provide an aerosolized active ingredient to a user may not have a lit-end. These articles may not burn during use. The upstream end of an inhaler article structured and arranged for engagement with a holder to provide an aerosolized active ingredient may have an upstream element that provides the upstream end face of the inhaler article. The upstream end face may be indistinguishable from the downstream end face of these inhaler articles, both before and after use.

For example, inhaler articles may contain active ingredient in dry powder form. If a dry powder active ingredient is contained in a capsule inside the inhaler article, the dry powder may be released when the inhaler article is engaged with a holder, the inhaler article is activated (by piercing the capsule, for example), and a user's puff initiates an airflow through the holder and through the inhaler article to entrain the dry powder active ingredient released from the capsule into the airflow, to deliver aerosolized dry powder active ingredient to the lungs of a user.

Or, for example, an inhaler article may contain aerosol-generating substrate. During use, when the inhaler article containing an aerosol-generating substrate is engaged with a holder, the inhaler article may be activated by heating the aerosol-generating substrate, which releases volatile compounds from the aerosol-generating substrate, entrains the volatile compounds into an airflow, and delivers aerosolized volatile active ingredients to the lungs of a user. Aerosol-generating substrate may be, for example, tobacco.

In both these examples, the inhaler article itself may have an upstream end and a downstream end which appear the same to the user before use. The inhaler article may have an upstream end face and a downstream end face which look similar. That is, before use, the upstream end and the downstream end may look the same. In both these examples, the inhaler article itself may have an upstream end and a downstream end which appear the same to the user after use. That is, after use, the upstream end and the downstream end may look the same.

In both these examples, the upstream end, the end that is inserted into the holder, may appear the same before and after use. That is, the user may not be able to determine whether the inhaler article is new or used by looking at the upstream end face of the inhaler article after use. And, in both of these examples, the downstream end, the mouthpiece end, may appear the same before and after use. That is, the user may not be able to determine whether the inhaler article is new or used by looking at the downstream end of the inhaler article after use.

Often, inhaler articles are packaged into a “pack” for sale to a user. For example, 10, 20 or more individual inhaler articles may be stacked together in a box so that multiple inhaler articles may be purchased together by the user in a pack. Often, multiple inhaler articles are provided in a pack with the downstream end inserted into the pack and the upstream end face of the inhaler article is visible to the user when the user opens the pack. Or, multiple inhaler articles may be provided in a pack with the upstream end inserted into the pack and the downstream end face of the inhaler article visible to the user when the user opens the pack.

Often, to reduce pollution, a user may remove an inhaler article from a pack, use the inhaler article, and then return the used inhaler article to the pack. By returning the used inhaler article to the pack instead of discarding the used inhaler article into the environment, the user reduces pollution. It is desirable to reduce pollution. However, when the user opens the pack to remove the next inhaler article from the pack, the user may not be able to distinguish between a new and a used inhaler article. If the user can't tell the difference between new inhaler articles contained in the pack and used inhaler articles returned to the pack, the next time the user removes an inhaler article from the pack, the user may remove a used article by mistake. A used inhaler article may not provide a satisfying experience when the user uses the inhaler article. If the user can't tell the difference between a new inhaler article and a used inhaler article that has been returned to the pack, the user may be less likely to return used inhaler articles to the pack. If the user is less likely to return a used inhaler article to the pack, the user is more likely to discard the used inhaler article into the environment.

Therefore, to reduce pollution, it is desirable to provide an inhaler article which can allow the user to see a difference between a new and a used inhaler article contained in a pack. One way to indicate to the user whether an inhaler article contained in a pack is a new inhaler article or a used inhaler article is to provide indicia on either the upstream end face or the downstream end face, or different indicia on both the upstream end face and the downstream end face. By providing indicia one or both the upstream end face or the downstream end face, the user can replace a used inhaler article into a pack in the opposite orientation from the new articles in the pack. When a used inhaler article has indicia on the upstream end face, the downstream end face or both, and the used inhaler article is replaced in a pack in an opposite orientation from the new articles that are in the pack, the replaced inhaler article will look different from the unused inhaler articles in the pack. The user will be able to tell, from looking at the end faces of the inhaler articles in the pack, which of the inhaler articles are new and which of the inhaler articles are used. When the user removes the next inhaler article from the pack, the user can tell which of the inhaler articles in the pack are unused and which of the inhaler articles in the pack are used. The user can remove an unused inhaler article from the pack because the user can see, at a glance, because of the presence of indicia, which inhaler articles in the pack are unused.

For example, when a used inhaler article is returned to a pack, if the inhaler article has indicia on one end face, and the user returns the used inhaler article to the pack in the opposite orientation from the new inhaler articles which have not been removed from the pack, the user can see by looking at the end faces in the pack, which of the inhaler articles contained in the pack have been used and which have not been used. In that way, using visual indicia, the user can distinguish between a new inhaler article and a used inhaler article that has been returned to the pack. If the user can distinguish between a new and a used inhaler article, the user is more likely to return used inhaler articles to the pack. If the user is more likely to return used inhaler articles to the pack, the user is less likely to dispose of the used inhaler article into the environment. Therefore, providing an inhaler article which has indicia on one end face is likely to reduce pollution of the environment. Therefore, to reduce pollution, it is desirable to provide indicia on an end face of an article.

In embodiments, the indicia is colored indicia. In embodiments, the indicia is one or more symbols. An indicia is something that is discernible or perceivable by the human eye. “Colored indicia” means a color that is different from the color of the inhaler article itself. That is, “colored indicia” can be seen against the background of the inhaler article. For example, if the inhaler article is white, the colored indicia is a color different from white. That is because if the indicia is the same color as the color of the inhaler article, the colored indicia will not be visible or perceivable by the user. “Colored indicia” is visible on the inhaler article. “Colored indicia” can include a surface of an inhaler article that is a color different from the color of the inhaler article. “Colored indicia” may be a single color (that is different from the color of the inhaler article). Or, “colored indicia” may include more than one colors that are different from the color of the inhaler article. Or, colored indicia may be in the form of a symbol. A symbol may be a letter. A symbol may be a series of letters. A symbol may be a word. A symbol may be a shape. A symbol may be an emoji. A symbol may be one color. A symbol may be multiple colors. Similarly, a symbol, to be visible, may be a different color than the inhaler article. If a symbol is the same color as the color of the inhaler article, the colored indicia will not be visible. For example, “colored indicia” may comprise a color such as blue, red, green, purple, yellow, black, gray yellow or orange or any color that is different from the inherent color of the inhaler article. When the inhaler article is white, the colored indicia is any color other than white. When the inhaler article is a color different from white, the indicia may be white, because a white indicia will be visible on a non-white inhaler article. In embodiments the indicia is one or more colors that indicate origin of the inhaler article. In embodiments, the indicia is one or more colors that indicate flavor of the inhaler article. In embodiments, the indicia is one or more colors that indicate strength or dosage for the inhaler article. In embodiments the indicia is one or more symbols that indicate origin of the inhaler article. In embodiments, the indicia is one or more symbols that indicate flavor of the inhaler article. In embodiments, the indicia is one or more symbols that indicate strength or dosage for the inhaler article. In embodiments, the indicia is a combination of color and symbols. In embodiments, the indicia is one or more colored symbols. In embodiments, the colored indicia is one or more symbols that indicate origin of the inhaler article. In embodiments, the colored indicia is one or more symbols that indicate flavor of the inhaler article. In embodiments, the colored indicia is one or more symbols that indicate strength or dosage for the inhaler article.

Inhaler articles may contain an active ingredient between the downstream element and the upstream element. The active ingredient may be, for example, tobacco or tobacco-related products or dry powder active ingredients. Dry powder active ingredients may be contained in a capsule in the inhaler article.

In embodiments, the indicia is provided on the upstream end face of the inhaler article. Because the upstream end of the inhaler article is upstream of the active ingredient, the active ingredient does not come into contact with the upstream end face during use. In contrast, the active ingredient entrained in the airflow passes through the downstream end face. Therefore, providing indicia on the upstream end face would be desirable to prevent the active ingredient from contacting the indicia and potentially delivering the indicia material to the user.

The present disclosure provides inhaler articles for use in inhaler systems that have indicia on an upstream end surface. It is possible to place indicia on an upstream end surface when the inhaler article has an upstream element which provides an upstream end surface capable of displaying indicia. In embodiments, it is possible to place indicia on an upstream end surface when the upstream end surface is present before and after use of the inhaler article. That is, in embodiments, when using the inhaler article destroys the upstream end surface, it is not possible to provide indicia on the upstream end surface after use of the inhaler article.

It is desirable to provide inhaler articles for use in inhaler systems that have colored indicia on the upstream end face, to assist the user to distinguish between new and used inhaler articles. It is desirable to provide inhaler articles for use in inhaler systems that have colored indicia on the upstream end face before and after use of the inhaler article. It is desirable to provide inhaler articles for use in inhaler systems that have colored indicia on the upstream end face that is visible to the user before and after use of the inhaler article.

In addition, it is desirable to provide inhaler articles for use in inhaler systems that have colored indicia on the upstream end face to provide information to the user. Such information may include information about origin of the inhaler article, flavor, strength, or other information.

The present disclosure provides inhaler articles for insertion into a holder to form an inhaler system for providing a compound to the lungs of a user, the inhaler article comprising a tubular body extending along a longitudinal axis from a downstream mouthpiece end to an upstream end. The upstream end has an upstream element. The upstream element has an upstream end surface. The upstream end surface comprises colored indicia.

These inhaler articles may be used in an inhaler system that provides dry powder to the lungs of a user by releasing dry powder contained in the inhaler article and entraining that dry powder into an airflow through the inhaler system for delivery to the lungs of a user. These inhaler articles may be used in a system that provides aerosol released from an aerosol-generating substrate by heating the aerosol-generating substrate to release an active ingredient into an airflow through the inhaler system for delivery to the lungs of a user. An inhaler article having an upstream element, wherein the upstream element has an upstream end surface comprising indicia can be used in both of these inhaler systems.

Two embodiments of inhaler articles having an upstream element that provides an upstream end face suitable for colored indicia are provided herein. However, those of ordinary skill in the art will recognize that any inhaler article having an upstream end face suitable for colored indicia will fall within the scope of this disclosure. And, in addition, inhaler articles having an upstream element that provide an upstream end face suitable for colored indicia before and after use will fall within the scope of this disclosure.

### Inhaler Articles for Dry Powder Delivery

It is desirable to provide inhaler articles for use in inhaler systems designed to deliver dry powder to a user, wherein the inhaler articles have an upstream element structured to reduce or minimize leakage of dry powder or loss of a capsule containing dry powder before and after use. For example, it is desirable to provide inhaler articles containing active ingredient in the form of dry powder having an upstream element with an upstream element end face which is closed before and after use.

Dry powder inhaler articles are not always fully suitable for providing airflow which is optimized for dry powder drug delivery during use, while also minimizing leakage of dry powder or loss of capsules from the inhaler articles before and after use.

It is desirable to provide a dry powder inhaler article which has an upstream element which closes the upstream end of the dry powder inhaler article before and after use and is also capable of opening during use. It is desirable to provide a dry powder inhaler article which is sufficiently closed before and after use to prevent loss of capsules contained in the dry powder inhaler article. It is desirable to provide a dry powder inhaler article which is sufficiently closed before and after use to prevent loss of dry powder contained in a capsule contained in the dry powder inhaler article. It is desirable to provide a dry powder inhaler article which is sufficiently open during use to allow optimal airflow through the dry powder inhaler article during use. It is desirable to provide a dry powder inhaler article having an upstream resilient element that is closed before and after use and is open during use. It is desirable to provide a dry powder inhaler article having an upstream element that is closed before and after use, and which has colored indicia to provide information to the user. The colored indicia may include color. The colored indicia may include at least one symbol. The colored indicia may include color and at least one symbol.

The inhaler article for insertion into a holder to form an inhaler system for providing a compound to the lungs of a user may include a tubular body extending along a longitudinal axis from a downstream mouthpiece end to an upstream end, the upstream end comprising an upstream element, the upstream element comprising an upstream end surface, wherein the upstream end surface comprises indicia. The indicia may be colored indicia. The indicia may be at least one symbol. The indicia may be a combination of colored indicia and at least one symbol. The indicia may be at least one colored symbol. The inhaler article may be a tubular article having an upstream end and a downstream end. The upstream end may be inserted into the holder. The downstream end, the mouth end, may be engaged by the mouth of a user to enable inhalation of the dry powder into the lungs of a user.

In an embodiment, the inhaler article contains a dose, or multiple doses, of dry powder active ingredient. The dry powder may be contained in a capsule inside the inhaler article. The inhaler article may be a disposable article. To use the inhaler system, a user may insert an inhaler article into the holder, activate the system to release dry powder from the capsule, inhale dry powder, and then remove the spent inhaler article from the holder. Activating the system may include piercing the capsule. In embodiments, the active ingredient is nicotine.

It is desirable that the inhaler article is structured and arranged to optimize dry powder delivery from the inhaler system to the lungs of a user during use. In addition, before and after use, it is desirable to ensure that the capsule, and the dry powder active ingredient, are safely contained in the inhaler article. That is, it is desirable that the capsule does not fall out of the inhaler article, before or after use. And, it is desirable that the dry powder contained in the capsule does not fall out of the inhaler article, before or after use. In addition, it is desirable that the upstream end surface of the inhaler article comprises colored indicia so that the user can confirm information about the inhaler article before and after use. Such information includes, but is not limited to origin, flavor or strength of the inhaler article. In addition, it is desirable to use colored indicia on the upstream end of the inhaler article to distinguish between a new and used inhaler article when the user replaces a used inhaler article into a pack of inhaler articles. For example, if the user replaces a used inhaler article into a pack in an orientation different from the new articles contained in the pack, the user can use the colored indicia to distinguish between new and used inhaler articles when the user chooses an inhaler article to use. For example, an inhaler article has colored indicia on the upstream end of the inhaler article, and does not have colored indicia on the downstream end of the inhaler article. All of the inhaler articles are initially placed into the pack in the same orientation. That is, the upstream end of all of the inhaler articles are visible when a user opens the pack. Or, the downstream end of all of the inhaler articles are visible when a user opens the pack. Then, when the user removes one of the inhaler articles from a pack containing multiple inhaler articles, uses the inhaler article, and replaces the inhaler article into the pack containing multiple inhaler articles in the opposite orientation from the orientation of the unused inhaler articles, the user can readily see which of the inhaler articles in the pack have been used.

It is desirable that the upstream end surface comprising colored indicia is visible when the inhaler article is contained in a pack, the pack containing a plurality of inhaler articles. It is desirable that at least one upstream end surface comprising colored indicia of at least one inhaler article contained in the pack is visible when the pack is open. In embodiments, when a plurality of inhaler articles are arranged in a pack, at least one of the inhaler articles is arranged in the pack in an unused orientation. In embodiments, when a plurality of inhaler articles are arranged in a pack, at least one of the inhaler articles is arranged in a used orientation. In embodiments, when a plurality of inhaler articles are arranged in a pack, the inhaler articles are arranged in a pack in a mixture of an unused orientation and a used orientation.

The disclosure provides an inhaler article having a tubular body, wherein the tubular body has, in linear sequential arrangement, from the upstream end to the downstream mouthpiece end an upstream element, a capsule cavity containing a capsule, the capsule containing dry powder active ingredient and a blocker element. The upstream element has an upstream end surface. The upstream element may have a resilient element. The resilient element may form the upstream end surface of the upstream element. The resilient element may be applied to the upstream end of the upstream element to form the upstream end surface of the upstream element. The resilient element may be affixed to the upstream end of the tubular body. The resilient element may be affixed to the upstream end face of the upstream element of the tubular body.

The present disclosure provides an inhaler article having a resilient element at the upstream end of the inhaler article, where the resilient element is in a closed position before insertion into the holder, where the resilient element opens to an open position in response to an opening force applied to the resilient element when the inhaler article is inserted into the holder in use, and where the resilient element closes to a closed position upon removal of the inhaler article from the holder after use. In an aspect, the upstream end of the capsule cavity of the inhalation article may be a resilient element forming an openable closed end of the inhaler article. The present disclosure provides an inhaler article having a resilient element at the upstream end of the inhaler article where the resilient element comprises colored indicia.

The resilient element of the inhaler article of the present disclosure is closed before and after use. It may not be possible to distinguish between a new inhaler article that has a closed resilient element on the upstream end and a used inhaler article that has a closed resilient element on the upstream end by looking at the end face of resilient element on the upstream end of the inhaler article. It may be possible to distinguish between a new and a used inhaler article by looking at the end face of the resilient element on the upstream end of the inhaler article.

The resilient element of the present disclosure has the advantage that it ensures that the capsule in the inhaler article is contained in the inhaler article before use. The resilient element opens when inserted into the holder to provide an airflow path from the upstream end of the inhaler article to the downstream mouthpiece end of the inhaler article. As air moves through the inhaler article, dry powder released from the capsule is entrained in the airflow and is delivered to the mouthpiece end of the inhaler article to be inhaled by the user. That is, the resilient element is structured and arranged to open to optimize dry powder delivery from the inhaler system to the lungs of a user during use. After use, upon removal of the inhaler article from the holder, the resilient element closes to retain the capsule inside the inhaler article. It is advantageous for the inhaler article to open when inserted into the holder during use to provide sufficient airflow to deliver the pharmaceutically active agent to the lungs of a user. It is advantageous for the inhaler article to be closed before use to prevent the capsule from falling out of the inhaler article before use. It is advantageous for the inhaler article to be closed after use to prevent the capsule from falling out of the inhaler article after use. It is advantageous for the inhaler article to be sufficiently closed after use, after the inhaler article has been activated to prevent the capsule from falling out of the inhaler article after use. The resilient element allows for both opening and closing of the upstream end of the inhaler, both before and after use of the inhaler article. The resilient element can open to provide sufficient airflow, when engaged with the holder, to optimize dry powder delivery to the lungs of a user and can close to prevent the capsule from falling out of the inhaler before and after insertion of the inhaler article into the holder. The resilient element on the upstream end of the inhaler article provides both the advantage of enabling sufficient airflow and also protecting from unwanted loss of pharmaceutically active agents from the inhaler article before and after use.

The present disclosure provides an inhaler article for insertion into a holder to form an inhaler system for providing a dry powder to the lungs of a user. The inhaler article may be a tubular body extending along a longitudinal axis from a downstream mouthpiece end to an upstream end; a capsule cavity within the tubular body between the downstream mouthpiece end and the upstream end; the capsule cavity containing a capsule. The upstream end of the inhaler article may have a resilient element. When inhaler article is inserted into the holder, the resilient element opens to an open position in response to an opening force provided by the holder. Upon removal of the inhaler article from the holder, the opening force is removed from the inhaler article. Upon removal of the opening force from the inhaler article, the resilient element closes to a closed position, sufficiently closed to retain the capsule in the capsule cavity of the inhaler article between the resilient element at the upstream end and the downstream mouthpiece end. The resilient element is made from elastic material suitable for opening and closing as described. The resilient element comprises colored indicia.

According to an aspect of the present disclosure, the inhaler article may be a tubular body extending along a longitudinal axis from a downstream mouthpiece end to an upstream end, having a capsule cavity within the tubular body between the downstream mouthpiece end and the upstream end, the capsule cavity containing a capsule. In an aspect, the downstream mouthpiece end has a blocker element. In an aspect the blocker is a filter element. The blocker element functions to ensure that the capsule does not exit the tubular body from the downstream end, before, during and after use. When the blocker is a filter, the filter element ensures that the pharmaceutically active powder delivered to the lungs of the user is powder that is small enough to pass through the filter. This ensures that the pharmaceutically active powder is suitable for delivery to the lungs of the user.

According to an aspect, the resilient element is a disk. In an aspect, the resilient element is round. In an aspect, the resilient element is an applied resilient element. The resilient element may be affixed to upstream end of the inhaler article. The resilient element may be affixed to the upstream end of the inhaler article by any process suitable to affix a resilient element to an inhaler article including gluing, heat sealing, pressing, friction fitting, or other means. The resilient element may be affixed to the upstream end of the inhaler article by gluing. The resilient element may be affixed to the upstream end of the inhaler article by heat sealing. The resilient element may be affixed to the upstream end of the inhaler article by pressing the resilient element into the inhaler article. The resilient element may be affixed to the upstream end of the inhaler article by friction fit. The resilient element may be affixed to the upstream end of the inhaler article by any suitable means.

According to an aspect, the resilient element of the inhaler article is a resilient element that has flaps. The resilient element may be pre-cut to form flaps. Or, flaps may be formed by assembling together flap parts and forming a resilient element having flaps. The flaps of the resilient element fold back when inserted into the holder to provide an opening which forms an airflow path from the upstream end of the inhaler article to the downstream mouthpiece end of the inhaler article. As air moves through the inhaler article, dry powder released from the capsule is entrained in the airflow and is delivered to the mouthpiece end of the inhaler article to be inhaled by the user. That is, the flaps of the resilient element open to form a relatively large airflow aperture to optimize dry powder delivery from the inhaler system to the lungs of a user during use. This relatively large airflow aperture allows for the formation of swirling airflow through the inhaler system, and allows maximum release of powder from the capsule and maximum delivery of powder to the user. After use, upon removal of the inhaler article from the holder, the resilient element closes to retain the capsule inside the inhaler article. The resilient element may be pre-cut to form at least 4 flaps. The resilient element may be pre-cut to form at least 6 flaps. The resilient element may be pre-cut to form at least 8 flaps. The cuts may extend between about 65% to about 95% of the diameter of the resilient element. Resilient material, provided at the upstream end of the inhaler article in the form of flaps, may open and close in a manner suitable to provide an open inhaler article during use when inserted into a holder and be closed before and after use to prevent the capsule from falling out of the inhaler article.

In an aspect, the flaps may open to an open position by folding the pre-cut flaps into the capsule cavity of the inhaler article in response to an opening force. The opening force may be provided by the holder. That is, a portion of the holder may fit inside the upstream end of the inhaler article when the inhaler article is inserted into the holder. As the portion of the holder fits inside the inhaler article, the holder may force the flaps to fold into the capsule cavity. When the flaps are folded into the capsule cavity of the inhaler article, an aperture is opened, and an airflow path is created. The resilient element having pre-cuts forming flaps opens to an open position by folding the pre-cut flaps into the capsule cavity in response to an opening force. Inserting the inhaler article into the holder may provide an opening force which forces the flaps of the pre-cut resilient element into the capsule cavity of the inhaler article.

In an aspect, the resilient element of the inhaler article is a resilient element that has a central aperture which opens to an open position by stretching open in response to an opening force. Stated another way, in an aspect, the resilient element of the inhaler article is an annular resilient element of resilient material which opens to an open position by stretching open in response to an opening force. The central aperture diameter may be less than 30% of the resilient element diameter. The opening force may be provided by the holder. The opening force may be provided by the movement of prongs of the holder into the central aperture of the upstream end of the inhaler article when the inhaler article is inserted into the holder. Inserting the inhaler article into the holder may provide an opening force which forces the central aperture to stretch open in response to the opening force.

The resilient element of the inhaler article may be made of resilient material. The resilient element of the inhaler article may be made of, for example, silicon, latex, plastic, paper, paper tape, laminated layered PLA on a paper layer, or cardboard. The resilient element may be made of silicon, latex, rubber or a combination. The resilient element of the inhaler article may be made of, for example, silicon. The resilient element of the inhaler article may be made of, for example, latex. The resilient element of the inhaler article may be made of, for example, plastic. The resilient element of the inhaler article may be made of, for example, paper. The resilient element of the inhaler article may be made of, for example, aluminium foil. The resilient element of the inhaler article may be made of, for example, paper tape. The resilient element of the inhaler article may be made of, for example, laminated layered PLA on a paper layer. The resilient element of the inhaler article may be made of, for example, cardboard. The resilient element of the inhaler article may be made of, for example, rubber. The resilient element of the inhaler article may be made of, for example, a combination of materials. The resilient element of the inhaler article may be made of, for example, any suitable resilient material.

In an aspect, when the resilient element is in the open position, engaged in the holder, the resulting opening or aperture provides unrestricted, or less restricted airflow into the capsule cavity. In an aspect, the holder is configured to provide swirling or rotational inhalation airflow to the inhaler article. In an aspect, the holder comprises a housing defining a housing cavity, a moveable cap configured to retain the inhaler article within the housing cavity, the moveable cap is movable within the housing cavity along the longitudinal axis of the housing, wherein the moveable cap comprises prongs and a piercing end. Prongs of the moveable cap extend into the resilient element at the upstream end of the inhaler article when the inhaler article is engaged in the holder. A piercing element, affixed to the inner surface of the bottom of the holder extends through the piercing end of the moveable cap. When the moveable cap moves in relation to the piercing element, the inhaler article is moved so that it is presented to the piercing element. The piercing element then pierces the capsule in the inhaler article to release pharmaceutically active dry powder from the capsule.

In an aspect, the resilient element has indicia. In embodiments, the indicia is colored indicia. In embodiments, the indicia is one or more symbols. In embodiments the indicia is one or more colors that are different from the color of the inhaler article, that indicate origin of the inhaler article. In embodiments, the indicia is one or more colors that are different from the color of the inhaler article that indicate flavor of the inhaler article. In embodiments, the indicia is one or more colors that are different from the color of the inhaler article that indicate strength or dosage for the inhaler article. In embodiments the indicia is one or more symbols that are a different color from the color of the inhaler article that indicate origin of the inhaler article. In embodiments, the indicia is one or more symbols that are a different color from the color of the inhaler article that indicate flavor of the inhaler article. In embodiments, the indicia is one or more symbols that are a different color from the color of the inhaler article that indicate strength or dosage for the inhaler article. “Color” may be any color. In order to be an indicia, the color must be different from the color of the inhaler article. That is, if indicia is not visible against the background of the inhaler article, it cannot be seen. The indicia must be visible in order to be useful. “Color” includes the color white. Colored indicia can be white if the background color to which the indicia is applied is not white. In embodiments, the indicia is a combination of color and symbols. In embodiments, the indicia is one or more colored symbols.

Disclosed herein is an inhaler article for insertion into a holder to form an inhaler system for providing a compound to the lungs of a user, the inhaler article comprising a tubular body extending along a longitudinal axis from a downstream mouthpiece end to an upstream end, the upstream end comprising an upstream element, the upstream element comprising an upstream end surface, wherein the upstream end surface comprises colored indicia. The upstream element may be a resilient element.

In an aspect, the capsule contains pharmaceutically active dry powder. The pharmaceutically active dry powder may be nicotine.

In dry powder inhaler systems, the systems are used to provide pharmaceutically active dry powder to the lungs of a user. The present disclosure provides a system and in particular an inhaler article, structured and arranged to contain active dry powder before and after use, and allow dry powder to leave, or be delivered from the inhaler article during use. The inhaler article has two ends, an upstream end and a downstream end. The downstream end is the mouthpiece end. The upstream end has an upstream end face which has colored indicia. During use, the user puts the downstream end of the inhaler article in the user's mouth and inhales.

The upstream end of the inhaler article is inserted into a holder during use. When the upstream end of the inhaler article is inserted into the holder, the upstream end of the inhaler article opens. When the upstream end of the inhaler article is inserted into the holder, the inhaler article plus the holder form an inhaler system. The inhaler article is inserted into the holder during use. The upstream end of the inhaler article opens during use to allow air to flow through the inhaler article and release pharmaceutically active dry powder to the user. The upstream end of the inhaler article opens when the inhaler article is inserted into the holder. The holder provides an opening force to the inhaler article to open the inhaler article to release pharmaceutically active powder from the inhaler article during use.

To prevent the pharmaceutically active dry powder from falling out of the inhaler article before use, the upstream end of the inhaler article is closed before use. After use, after the inhaler article is removed from the holder, the upstream end of the inhaler article closes to prevent the pharmaceutically active dry powder from falling out of the inhaler after use. The upstream end of the inhaler article is open when it is inserted into a holder and closed before and after the inhaler article is inserted into the holder.

To enable the upstream end of the inhaler article to be closed before use, open during use, and then closed again after use, the upstream end of the inhaler article is a resilient element. The resilient element is closed before use, open during use, and then comes back to a closed position after use. The resilient element is closed before use, opens when the inhaler article is inserted into the holder, and then closes again when the inhaler article is removed from the holder. The resilient element may be colored, or may have one or more symbols, or may have both color and one or more symbols. Color and symbols are indicia. These indicia, present alone or in combination, convey information to the user.

The inhaler article may be any shape or size. For ease of insertion into a holder, the inhaler article may be a tubular article. The inhaler article may be a tubular body extending along a longitudinal axis from a downstream mouthpiece end to an upstream end. The inhaler article may be cylindrical. The inhaler article contains pharmaceutically active powder.

The inhaler article may resemble a smoking article or cigarette in size and shape. The inhaler article may be a tubular body extending along the longitudinal axis of the inhaler article. The inhaler article may have a substantially uniform outer diameter along the length of the elongated body. The inhaler body may have a circular cross-section that may be uniform along the length of the elongated body. The inhaler body may have an outer diameter in a range from about 6 mm to about 10 mm, or from about 7 mm to about 10 mm, or about 7 mm to about 9 mm, or about 7 mm to about 8 mm or about 7.2 mm. The inhaler body may have a length (along the longitudinal axis) in a range from about 40 mm to about 80 mm, or from about 40 mm to about 70 mm, or about 40 mm to about 50 mm, or about 45 mm.

The inhaler article may be a tubular body extending along a longitudinal axis from a downstream mouthpiece end to an upstream end. Between the downstream mouthpiece end and the upstream end is a central cavity.

The downstream end of the inhaler article is the mouthpiece end. That is, the downstream end of the inhaler article is intended to engage with the mouth of a user to allow the contents of the inhaler article to be inhaled by a user. The downstream end is structured and arranged for engagement with the mouth of a user. The downstream end is sized and shaped for engagement with the mouth of a user. The downstream end of the inhaler article acts as a conduit to provide pharmaceutically active powder to the mouth and lungs of a user. The downstream end of the inhaler article has a stiffness or hardness, a size, and a shape for engagement with the mouth of a user. The downstream end of the inhaler article may have a blocker. The blocker may be an element of the inhaler article that provides a stiffness or hardness. The stiffness or hardness of the blocker is appropriate for insertion into the mouth of a user during use of the inhaler system. The blocker may have a size and a shape for engagement with the mouth of a user.

In addition, the blocker may be a filter element. The filter element may have an internal structure that allows smaller particles to flow through the filter element and blocks larger particles from flowing through the filter element. The filter element may act to filter larger particles of pharmaceutically active powder as they flow out of the inhaler article. The filter element may act to control the size of particles of pharmaceutically active powder as they flow out of the inhaler article. The filter element may allow particles of pharmaceutically active powder to flow out of the inhaler article to a user. The filter element may allow particles of pharmaceutically active powder that are small enough to flow through the filter element out of the inhaler article to a user. The filter element may be formed of a cellulose acetate material. The filter element may be cellulose tow. The filter may be formed of a biodegradable material.

The blocker element may extend from the central cavity to the mouthpiece end of the inhaler article. The blocker element may extend from the capsule cavity to the mouthpiece end of the inhaler article. The filter element may have a length in a range from about 10 mm to about 30 mm, preferably from about 15 mm to about 25 mm and more preferably from about 20 mm to about 22 mm.

The inhaler article has an upstream end. The upstream end has an upstream element. The upstream element may be a resilient element. The resilient element of the inhaler article may have cuts which allow the resilient element to open when prongs are inserted into the resilient element. These cuts may be referred to as cuts or pre-cuts. These cuts can be described as radial cuts or diametric cuts. Two diametric cuts, results in the formation of four flaps in the resilient element. Two diametric cuts, extending partially across the diameter of the resilient element, is the same as four radial cuts, extending partially across the radius of the resilient element. The cuts may extend in a range from about 65% to about 95% of the diameter of the resilient element. The cuts may extend in a range from 65% to 95% of the diameter of the resilient element. Alternatively, flaps may be manufactured by assembling parts together to form flaps.

When the inhaler article having a resilient element having cuts (and flaps) is inserted into a holder, the prongs of the holder push the flaps into the inside of the inhaler article. When the prongs of the holder push the flaps into the inside of the inhaler article, the inhaler article is open. The prongs of the holder provide an opening force to open the upstream end of the inhaler article. When the inhaler article is open, an airflow passage is provided which improves airflow through the inhaler system. When the inhaler article is withdrawn from the holder, the prongs are withdrawn from the inhaler article and the flaps return to their position at the upstream end of the inhaler article. When the opening force is withdrawn, the inhaler article closes. When the inhaler article is removed from the holder, the opening force is removed from the upstream end of the inhaler article. When the opening force is removed from the inhaler article, the resilient element returns to a closed position. Upon removal of the opening force, the resilient element closes to a closed position. While the resilient element having a cuts and flaps may not be fully closed before or after use, it is partially closed before and after use. The resilient element is not open after use.

The resilient element of the inhaler article may have a central aperture which opens to an open position by stretching open in response to an opening force. Stated another way, in an aspect, the resilient element of the inhaler article is an annular resilient element of resilient material which opens to an open position by stretching open in response to an opening force. The central aperture diameter may be less than 30% of the resilient element diameter. The opening force may be provided by the holder. The opening force may be provided by the movement of prongs of the holder into the central aperture of the upstream end of the inhaler article when the inhaler article is inserted into the holder. Inserting the inhaler article into the holder may provide an opening force which forces the central aperture to stretch open in response to the opening force. In addition, the resilient element returns to a closed position when the opening force is removed. When the inhaler article is removed from the holder, the opening force is removed from the upstream end of the inhaler article. When the opening force is removed from the inhaler article, the resilient element returns to a closed position. Upon removal of the opening force, the resilient element closes to a closed position. While the resilient element having a central aperture may not be fully closed before or after use, it is partially closed before and after use. It is not open after use.

The resilient element of the inhaler article may have a combination of cuts and a central aperture which opens to an open position by stretching open in response to an opening force. Stated another way, in an aspect, the resilient element of the inhaler article is an annular resilient element of resilient material which opens to an open position by stretching open in response to an opening force. The central aperture diameter may be less than 30% of the resilient element diameter. The cuts may extend in a range of 65% to 95% of the diameter of the resilient element. The opening force may be provided by the holder. The opening force may be provided by the movement of prongs of the holder into the central aperture of the upstream end of the inhaler article when the inhaler article is inserted into the holder. Inserting the inhaler article into the holder may provide an opening force which forces the central aperture to stretch open in response to the opening force.

When the inhaler article is removed from the holder, the prongs are removed from the inhaler article and the resilient element closes. When the inhaler article is removed from the holder the opening force is removed. When the prongs are removed from the inhaler article, the opening force is removed. When the opening force is removed the resilient element closes. When the inhaler article is removed from the holder, the opening force is removed from the upstream end of the inhaler article. When the opening force is removed from the inhaler article, the resilient element returns to a closed position. Upon removal of the opening force, the resilient element closes to a closed position. Upon removal from the holder, the colored indicia on the upstream end surface of the closed resilient element is visible.

Between the downstream end and the upstream end of the tubular body is a central cavity which contains pharmaceutically active powder. The central cavity may have a length in a range from about 3 mm to about 12 mm, or from about 3 mm to about 7 mm or about 4 mm to about 6 mm, or about 5 mm.

The cavity may contain pharmaceutically active powder contained in a capsule. When the cavity contains pharmaceutically active powder contained in a capsule, the cavity is a capsule cavity.

The capsule cavity may be the inside of the inhaler article between the downstream blocker element and the upstream resilient element. The downstream blocker element may be joined with the upstream resilient element by a wrapper. The wrapper may form the tubular body of the inhaler article. The tubular body defining the capsule cavity may be formed of a biodegradable material, such as cardboard or paperboard.

The capsule cavity may have an inner diameter in a range from about 6 mm to about 7 mm, or about 6.5 mm to about 6.7 mm. The capsule cavity may have a lateral length in a range from about 15 mm to about 30 mm, or from about 20 mm to about 25 mm.

The capsule cavity may define a cylindrical space configured to contain a capsule (the capsule may have an obround shape or a circular cross-section, for example). The capsule cavity may have a substantially uniform or uniform diameter along the length of the capsule cavity. The capsule cavity may have a fixed cavity length. The capsule cavity has a cavity inner diameter, orthogonal to the longitudinal axis, and the capsule has a capsule outer diameter. The capsule cavity may be sized to contain an obround capsule. The capsule cavity may have a substantially cylindrical or cylindrical cross-section along the length of the capsule cavity. The capsule cavity may have a uniform inner diameter. The capsule may have an outer diameter that is about 80% to about 95% of the inner diameter of the capsule cavity. The configuration of the capsule cavity relative to the capsule may promote limited movement of the capsule during activation or piercing of the capsule.

The configuration of the capsule cavity relative to the capsule may promote the capsule to rotate with stability within the capsule cavity. The longitudinal axis of the capsule may rotate with stability co-axially with the longitudinal axis of the inhaler body during inhalation. The configuration of the capsule cavity relative to the capsule may promote the capsule to rotate with some shaking within the capsule cavity.

Stable rotation refers to the longitudinal axis of the inhaler body being substantially parallel or co-axial with the axis of rotation of the capsule. Stable rotation may refer to the absence of procession of the rotating capsule. Preferably the longitudinal axis of the inhaler body may be substantially coextensive with the axis of rotation of the capsule. Stable rotation of the capsule may provide a uniform entrainment of a portion of nicotine particles from the capsule over two or more, or five or more, or ten or more “puffs” or inhalations by a consumer.

The capsule may be contained within the inhaler article prior to consumption. The inhaler article may be contained within the capsule cavity by the resilient element.

The capsule may be formed of an airtight material that may be pierced or punctured by a piercing element that may be separate or combined with the inhaler. The capsule may be formed of a metallic or polymeric material that serves to keep contaminates out of the capsule but may be pierced or punctured by a piercing element prior to consumption of the nicotine particles within the capsule. The capsule may be formed of a polymer material. The polymer material may be hydroxypropylmethylcellulose (HPMC). The capsule may be a size 1 to size 4 capsule, or a size 3 capsule.

The capsule contains pharmaceutically active particles. The pharmaceutically active particles may comprises nicotine (also referred to as “nicotine powder” or “nicotine particles”) and optionally particles comprising flavour (also referred to as “flavour particles). The capsule may contain a predetermined amount of nicotine particles and optional flavour particles. The capsule may contain enough nicotine particles to provide at least 2 inhalations or “puffs”, or at least about 5 inhalations or “puffs”, or at least about 10 inhalations or “puffs”. The capsule may contain enough nicotine particles to provide from about 5 to about 50 inhalations or “puffs”, or from about 10 to about 30 inhalations or “puffs”. Each inhalation or “puff” may deliver from about 0.1 mg to about 3 mg of nicotine particles to the lungs of the user or from about 0.2 mg to about 2 mg of nicotine particles to the lungs of the user or about 1 mg of nicotine particles to the lungs of the user.

The nicotine particles may have any useful concentration of nicotine based on the particular formulation employed. The nicotine particles may have at least about 1% wt nicotine up to about 30% wt nicotine, or from about 2% wt to about 25% wt nicotine, or from about 3% wt to about 20% wt nicotine, or from about 4% wt to about 15% wt nicotine, or from about 5% wt to about 13% wt nicotine. Preferably, about 50 to about 150 micrograms of nicotine may be delivered to the lungs of the user with each inhalation or “puff”.

The capsule may hold or contain at least about 5 mg of nicotine particles or at least about 10 mg of nicotine particles. The capsule may hold or contain less than about 900 mg of nicotine particles, or less than about 300 mg of nicotine particles, or less than 150 mg of nicotine particles. The capsule may hold or contain from about 5 mg to about 300 mg of nicotine particles or from about 10 mg to about 200 mg of nicotine particles.

The nicotine particles may have any useful size distribution for inhalation delivery preferentially into the lungs of a user. The capsule may include particles other than the nicotine particles. The nicotine particles and the other particles may form a powder system.

The filter element may provide structure which allows particles having a desirable size to pass through, while preventing particles having larger size from passing through the filter. For example, the filter element may dry powder having a mean diameter from about 60 micrometres or less to pass through the filter element, while preventing dry powder having a mean diameter above about 60 micrometres from passing through. Or, if flavour particles are desired, the filter element may provide structure which allows particles having up to 200 micrometres to pass through the filter.

Nicotine in the powder system or nicotine particles may be a pharmaceutically acceptable free-base nicotine, or nicotine salt or nicotine salt hydrate. Useful nicotine salts or nicotine salt hydrates include nicotine pyruvate, nicotine citrate, nicotine aspartate, nicotine lactate, nicotine bitartrate, nicotine salicylate, nicotine fumarate, nicotine mono-pyruvate, nicotine glutamate or nicotine hydrochloride, for example. The compound combining with nicotine to form the salt or salt hydrate may be chosen based on its expected pharmacological effect.

The nicotine particles preferably include an amino acid. Preferably the amino acid may be leucine such as L-leucine. Providing an amino acid such as L-leucine with the particles comprising nicotine, may reduce adhesion forces of the particles comprising nicotine and may reduce attraction between nicotine particles and thus reduce agglomeration of nicotine particles. Similarly, adhesion forces to particles comprising flavour may also be reduced thus agglomeration of nicotine particles with flavour particles is also reduced. The powder system described herein thus may be a free-flowing material and possess a stable relative particle size of each powder component even when the nicotine particles and the flavour particles are combined.

The nicotine may be a surface modified nicotine salt where the nicotine salt particle comprises a coated or composite particle. A preferred coating or composite material may be L-leucine. One particularly useful nicotine particle may be nicotine bitartrate with L-leucine.

A holder for an inhaler article includes a housing comprising a housing cavity for receiving an inhaler article, a moveable cap to enable engagement of the inhaler article with a piercing element, a piercing element, prongs to insert into the upstream end of the inhaler article when the inhaler article is inserted into the holder, and air inlets and airflow passages to enable swirling airflow through the inhaler article when it is engaged in the holder. This swirling or rotational inhalation airflow may be transmitted into an inhaler article to rotate and agitate a capsule and release dry powder contained within the capsule. The holder has an open end and a piercing end. The housing cavity is sized to receive an inhaler article. The inhaler article is inserted into the open end.

The holder has a piercing element. The piercing element is affixed to the inside of the bottom surface of the holder. The piercing element length may be any suitable length relative to the housing length. For example, the piercing element length may be about 25% to about 60%, or about 30% to about 50%, of the housing length. A distal end of the piercing element may be fixed to the distal end adjacent to or at the distal end of the housing. The piercing element entire length may be coextensive within the housing length. The piercing element is formed of a rigid material. The rigid material is sufficiently rigid to pierce, puncture or activate a capsule contained within the inhaler article. The piercing element may be formed of a metal. The piercing element may be formed of stainless steel, such as 316 stainless steel, for example. The piercing element may be formed of a polymeric material. The piercing element may be formed of a fibre-reinforced polymeric material.

The piercing element extends through the moveable cap. The moveable cap is moveable in relation to the piercing element. When the inhaler article is inserted into the holder, and is pressed down, the moveable cap moves down into the holder, exposing the piercing element. When the inhaler article is pressed down against the moveable cap, the piercing element extends into the inhaler article and pierces the capsule. Piercing the capsule activates the capsule. Piercing the capsule activates the inhaler system. Piercing the capsule allows for the release of powder from the capsule.

When the inhaler article is inserted into the holder, a portion of the holder inserts into the upstream end of the inhaler article to open the upstream end of the inhaler article. When a portion of the holder inserts into the upstream end of the inhaler article, the holder provides an opening force to the inhaler article. In embodiments, the portion of the holder that inserts into the upstream end of the inhaler article is prongs. When the inhaler article is inserted into the holder, prongs of the holder insert into the upstream end of the inhaler article. When prongs of the holder insert into the upstream end of the inhaler article, the prongs provide an opening force to the inhaler article. When the prongs of the holder insert into the upstream end of the inhaler article, the upstream end of the inhaler article is opened. When the prongs of the holder insert into the upstream end of the inhaler article, an opening force is applied to the upstream end of the inhaler article. When the prongs of the holder insert into the upstream end of the inhaler article which is a resilient element, the resilient element opens. When the inhaler article is removed from the holder, the opening force is removed from the upstream end of the inhaler article. When the opening force is removed from the inhaler article, the resilient element returns to a closed position. Upon removal of the opening force, the resilient element closes to a closed position.

When the inhaler article is inserted into the holder, a portion of the holder inserts into the resilient element at the upstream end of the inhaler article to open the resilient element the inhaler article. When a portion of the holder inserts into the resilient element of the inhaler article, the holder provides an opening force to the inhaler article. When a portion of the holder inserts into the resilient element of the inhaler article, the holder provides an opening force to the resilient element. When a portion of the holder inserts into the upstream end of the inhaler article, the holder provides an opening force to the upstream end of the inhaler article. When the inhaler article is removed from the holder, the opening force is removed from the upstream end of the inhaler article. When the opening force is removed from the inhaler article, the resilient element returns to a closed position. Upon removal of the opening force, the resilient element closes to a closed position.

Prongs can be shaped and sized to optimize insertion into the upstream end of the inhaler article. Prongs can be shaped and sized to optimize insertion into the resilient element of the inhaler article. For example, when the resilient element has a central aperture, the prongs may be angled to optimize insertion into the central aperture and expansion of the resilient element. Two or more prongs may be present. For example, 2, prongs may be present. Three prongs may be present. Four prongs may be present. Five prongs may be present. Six prongs may be present. More than six prongs may present. Or, prongs may mean an annular ring which inserts into the upstream end of the inhaler article. Or, prongs may mean an annular ring which inserts into the resilient element of the inhaler article.

The housing may be formed of any rigid material. The housing may be formed of a polymeric material. Polymeric materials useful for forming the housing include polycarbonate, polypropylene, polyethylene, nylon, acrylonitrile butadiene styrene, styrene acrylonitrile, polyacrylate, polystyrene, PBT polyester, PET polyester, polyoxymethylene, polysulfone, polyethersulfone, polyethereetherketone, or liquid crystal polymer.

The present disclosure provides an inhaler article having pharmaceutically active dry powder contained in a capsule. Disclosed herein is an inhaler article containing a capsule, the capsule containing pharmaceutically active powder. In embodiments, the pharmaceutically active powder contains nicotine, although other pharmaceutically active powders are contemplated in this disclosure, as discussed below. The inhaler article can be used to deliver pharmaceutically active powder to a user when the user inhales from the mouthpiece end (the downstream end, the proximal end) of the inhaler article. In order to deliver pharmaceutically active dry powder that is contained in a capsule to the user, the pharmaceutically active powder is released from the capsule, the powder is aerosolized, and inhaled by the user. That is, the powder is released from the capsule and entrained into the airflow created by the user when the user inhales from the mouthpiece end (the downstream end, the proximal end) of the inhaler article.

According to the present disclosure, the inhaler article has a tubular body extending along a longitudinal axis from a downstream mouthpiece end to an upstream end. Inside of the tubular body is a capsule cavity. The capsule cavity contains a capsule containing pharmaceutically active powder.

In order to release the pharmaceutically active powder from the capsule contained in the capsule cavity of the inhaler article, the capsule must open in a way that allows the powder contained inside the capsule to be released. In an embodiment, the capsule is pierced. When the capsule is pierced, a hole is introduced into the capsule. This releasing step or piercing step may also be considered an “activation”. According to the present disclosure, the capsule is activated when it is pierced by a piercing element. In embodiments, the piercing element is located in a holder.

When the inhaler article is inserted into a holder, the upstream end of the inhaler article is inserted into the holder. While inserting the inhaler article into the holder, the inhaler article may press down against a piercing element located in the holder, and the capsule may be pierced by the piercing element. Once inserted, the downstream end of the inhaler article extends from the holder and is accessible to the mouth of a user. Once the capsule has been pierced or activated, powder contained in the capsule can be released from the capsule. Powder from the capsule can then be released into an airflow, and is inhaled by a user. The inhaler article is inserted into a holder to form an inhaler system, the capsule is pierced by a piercing element of the holder, an airflow is initiated by the user, the airflow passes through the inhaler article, entrains powder that has been released from the capsule, and the pharmaceutically active powder is delivered to a mouth of a user.

The holder is separate from the inhaler article, but the consumer may utilize both the inhaler article and the holder while consuming the particles released within the inhaler article. A plurality of these inhaler articles may be combined with a holder to form a system or kit. A single holder may be utilized on 10 or more, or 25 or more, or 50 or more, or 100 or more, inhaler articles to activate (puncture or pierce) a capsule contained within each inhaler article and provide reliable activation.

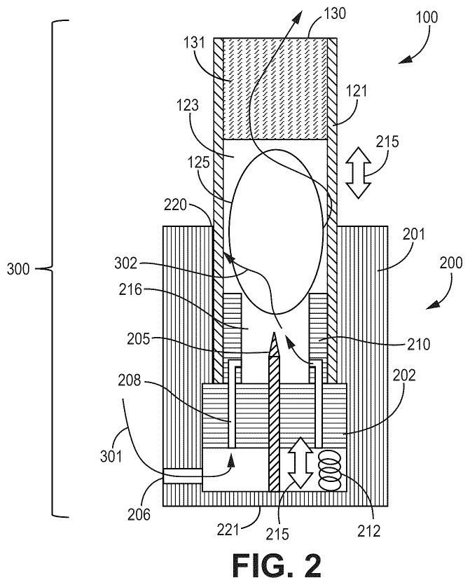

A holder for an inhaler article includes a housing comprising a housing cavity for receiving an inhaler article and configured to retain an inhaler article within the housing cavity. A moveable cap is in the housing cavity and is movable within the housing cavity along the longitudinal axis of the housing. The moveable cap has prongs. The prongs of the moveable cap are configured to insert into the inhaler article when the inhaler article is introduced into the holder. The moveable cap is configured to move in relation to a piercing element. When an inhaler article is introduced into the housing cavity of the holder, it is pressed down into the holder. As the inhaler article is pressed into the housing cavity, the prongs of the moveable cap insert into the upstream end of the inhaler article. At the same time, the inhaler article is pressed down into the housing against the moveable cap. The moveable cap moves down. The piercing element extends through the moveable cap as the moveable cap moves so that the capsule inside the inhaler article contacts and is pierced by the piercing element. The inhaler article is then released, and the moveable cap moves back to a resting position. The moveable cap may move back to a resting position by means of a spring. The position of the inhaler article, inserted into the holder, after the capsule has been pierced, is shown in FIG. 2 below.

The prongs of the moveable cap are able to insert into the upstream end of the inhaler article through the resilient element at the upstream end of the inhaler article.

A method includes, removing an unused inhaler article from a pack, where the unused inhaler article was in the pack in an unused orientation, inserting the inhaler article into the housing cavity of the holder to form the inhaler system, activating the inhaler article to entrain active ingredient into an airflow through the inhaler system, delivering active ingredient to the lungs of a user, removing the inhaler article from the holder, and replacing the inhaler article into the pack in an opposite orientation from the unused orientation. Then another unused inhaler article may be inserted into the holder and the method may be repeated.

The inhaler article associated with the holder described above is configured to receive swirling inhalation airflow directly into the distal end of the inhaler article. The swirling inhalation airflow is initiated when a user “puffs” on the downstream end of the inhaler article inserted into a holder. This creates negative pressure inside the inhaler article. As a result of this negative pressure, air enters the holder via an air inlet. Air then travels through air passages in the holder. The air passages in the holder direct air into the inhaler article at an angle tangential to the longitudinal axis of the inhaler article, when inserted into the holder. This tangential air creates swirling airflow in the inhaler article when inserted into the holder. This swirling airflow causes the capsule to rotate and/or agitate within the inhaler article. This agitation of the capsule improves the flow of powder out of the activated capsule, and improves the efficiency with which powder is entrained into airflow delivered to the user.

The resilient element may be made from any material suitable for opening and closing. For example, the resilient element may be made from silicon. The resilient element may be made from latex. The resilient element may be made from rubber. The resilient element may be made from plastic. The resilient element may be made from paper. The resilient element may be made from aluminium foil. The resilient element may be made from laminated layered PLA on a paper layer. The resilient element may be made from cardboard. The resilient element may be made from silicon, latex or rubber. The resilient element may be made from silicon, latex, rubber of a combination thereof.

The resilient element may be affixed to the inhaler article. The resilient element may be affixed to the inhaler article by any means. For example the resilient element may be affixed to the inhaler article by gluing, friction fitting, heat sealing, or by any means.

### Inhaler Article for Use in Heated Inhaler System

Also disclosed herein is an inhaler article for insertion into a holder to form an inhaler system for providing a compound to the lungs of a user, wherein the inhaler system includes a heater for generating aerosol from an aerosol-generating substrate. Such an inhaler system is disclosed in, for example, WO2021170667, incorporated herein in its entirety.

For example, in a heated inhaler system, the tubular body of the inhaler article comprises, in linear sequential arrangement, from the upstream end to the downstream mouthpiece end, an upstream element, a rod of aerosol-generating substrate located downstream of the upstream element, an aerosol-cooling element located downstream of the support element, a mouthpiece element located downstream of the aerosol-cooling element, and an outer wrapper circumscribing the upstream element, the aerosol-cooling element and the mouthpiece element. The upstream element has an upstream end face which has colored indicia.

As in the dry powder inhaler system, the heated inhaler system does not require that an end of the inhaler article is lit. Instead, the upstream end is inserted into a holder which provides heat to a rod of aerosol-generating substrate. In heated aerosol generating articles, an aerosol is generated by heating an aerosol-generating substrate, such as tobacco. Known heated aerosol generating articles include, for example, electrically heated aerosol generating inhaler articles and aerosol generating articles in which an aerosol is generated by the transfer of heat from a heat source to a physically separate aerosol forming material. When the aerosol-generating substrate is heated, in heated inhaler articles, an aerosol is generated by heating an aerosol-generating substrate, such as tobacco. For example, inhaler articles according to the invention find particular application in aerosol generating systems comprising an electrically heated aerosol generating device having an internal heater blade which is adapted to be inserted into the rod of aerosol generating substrate. In embodiments, the internal heater blade is inserted into the rod of aerosol generating substrate to heat the aerosol generating substrate by conduction. In embodiments, the internal heater blade is a susceptor which is heated by induction from a magnetic field generated in or by the holder. In embodiments, the susceptor is located in the rod of aerosol-generating substrate in the inhaler article.

Disclosed herein is an inhaler article for insertion into a holder to form an inhaler system for providing a compound to the lungs of a user, the inhaler article comprising a tubular body extending along a longitudinal axis from a downstream mouthpiece end to an upstream end. The upstream end comprises an upstream element. The upstream element provides a barrier between the active ingredient and the upstream end face of the inhaler article. The upstream element prevents contact with the aerosol-generating substrate. The upstream element may prevent the rod of aerosol-generating substrate from falling out of the inhaler article. The upstream element may prevent direct contact with a susceptor contained in the rod of aerosol-generating substrate. This helps to prevent the displacement or deformation of the susceptor element during handling or transport of the inhaler article. This in turn helps to secure the form and position of the susceptor element. The upstream element may prevent the susceptor contained in the rod of aerosol-generating substrate from falling out of the inhaler article.

The upstream element may be a porous plug element. Preferably, a porous plug element does not alter the resistance to draw of the aerosol-generating article. Preferably, the upstream element has a porosity of at least about 50 percent in the longitudinal direction of the aerosol-generating article. Or, in embodiments, the upstream element has a porosity of between about 50 percent and about 90 percent in the longitudinal direction. The porosity of the upstream element in the longitudinal direction is defined by the ratio of the cross-sectional area of material forming the upstream element and the internal cross-sectional area of the aerosol-generating article at the position of the upstream element. In embodiments, the upstream element may be formed from a material that is impermeable to air. In such embodiments, the aerosol-generating article may be configured such that air flows into the rod of aerosol-generating substrate through a suitable ventilation means provided in a wrapper. The upstream element may be made of any material suitable for use in an inhaler article. The upstream element may, for example, be made of the same material as used for one of the other components of the inhaler article, such as the blocker or filter. Suitable materials for forming the upstream element include filter material, ceramic, polymer material, cellulose acetate, cardboard or zeolite. The upstream element may be formed from a plug of cellulose acetate, for example.

The upstream element may be formed of a heat-resistant material. For example, the upstream element may be formed of a material that resists temperatures of up to 350 degrees Celsius. This ensures that the upstream element is not adversely affected by heating the aerosol-forming substrate. The upstream element may have a diameter that is approximately equal to the diameter of the inhaler article. The upstream element may have a substantially homogenous structure. The upstream element may have a continuous, regular surface over its entire cross-section.

When looking at the upstream end of the inhaler article, the active ingredient is not visible. Compared to a traditional smoking article, where tobacco is visible at the lit-end or the upstream end, here, the upstream end of the upstream element is visible at the upstream end of the inhaler article. The upstream end face of the inhaler article is available for marking with colored indicia.

The upstream element has an end face. The upstream element end face may have colored indicia. The upstream element end face may have color as indicia. To be visible, the color of the indicia should be different from the color of the inhaler article itself. The upstream element may have one or more symbols as indicia. The upstream element may have both color and one or more symbols as indicia. The upstream element may have one or more colored symbols as indicia.

A rod of aerosol-generating material may be plant material. The plant material may be tobacco. The tobacco may be present in the rod as homogenized plant material. Homogenised plant material can be provided in any suitable form. For example, homogenised plant material may be present as sheets or webs, pellets, particles, granules, strands, strips, shreds, or crimped material. Sheets may be gathered, folded, shredded, crimped, or corrugated. “Tobacco particles” includes ground or powdered tobacco leaf lamina, ground or powdered tobacco leaf stems, tobacco dust, tobacco fines and other particulate tobacco by-products formed during the treating, handling and shipping of tobacco. Different types of tobacco may be used, and these types of tobacco may be treated, cured, dried and blended to provide a preferred tobacco formulation for use in a rod of aerosol-generating substrate.

The aerosol-generating material may contain a compound. The aerosol-generating material may contain a pharmaceutically active compound. The aerosol-generating material may contain an active agent. In embodiments, the compound is nicotine. In embodiments, the active agent is nicotine.

The rod of aerosol-generating substrate may also contain an aerosol-former. This aerosol-former is volatilised upon heating and a stream of the aerosol former is contacted with the aerosol-generating substrate so as to entrain the flavours and active agents from the aerosol-generating substrate in the aerosol delivered to the user. In embodiments the aerosol-former is glycerol.

In embodiments, the rod of aerosol-generating substrate contains a susceptor. Preferably, the elongate susceptor has a length which is the same or shorter than the length of the rod of aerosol-generating substrate. Preferably, the elongate susceptor has a same length as the rod of aerosol-generating substrate. The susceptor may be formed from any material that can be inductively heated to a temperature sufficient to generate an aerosol from the aerosol-generating substrate. Preferred susceptors comprise a metal or carbon. A preferred susceptor may comprise or consist of a ferromagnetic material, for example a ferromagnetic alloy, ferritic iron, or a ferromagnetic steel or stainless steel. A suitable susceptor may be, or comprise, aluminium. Preferred susceptors may be formed from 400 series stainless steels, for example grade 410, or grade 420, or grade 430 stainless steel. Different materials will dissipate different amounts of energy when positioned within electromagnetic fields having similar values of frequency and field strength. Thus, parameters of the susceptor such as material type, length, width, and thickness may all be altered to provide a desired power dissipation within a known electromagnetic field. Preferred susceptors may be heated to a temperature in excess of 250 degrees Celsius. Suitable susceptors may comprise a non-metallic core with a metal layer disposed on the non-metallic core, for example metallic tracks formed on a surface of a ceramic core. A susceptor may have a protective external layer, for example a protective ceramic layer or protective glass layer encapsulating the susceptor. The susceptor may comprise a protective coating formed by a glass, a ceramic, or an inert metal, formed over a core of susceptor material. The susceptor is arranged in thermal contact with the aerosol-generating substrate. Thus, when the susceptor heats up, the aerosol-generating substrate is heated up and an aerosol is formed. Preferably the susceptor is arranged in direct physical contact with the aerosol-generating substrate, for example within the aerosol-generating substrate.

In embodiments, downstream of the rod of aerosol-generating substrate is an aerosol-cooling element. In embodiments, the aerosol-cooling element is a hollow section downstream from the rod of aerosol-generating substrate. The aerosol-cooling element may cool aerosol moving through the inhaler article by any means, including introducing external air into the aerosol-cooling element. The aerosol-cooling element is arranged substantially in alignment with the rod of aerosol-generating substrate. The aerosol-cooling element may be formed from any suitable material or combination of materials. For example, the aerosol-cooling element may be formed from one or more materials selected from the group consisting of: cellulose acetate; cardboard, crimped paper, such as crimped heat-resistant paper or crimped parchment paper; and polymeric materials, such as low density polyethylene (LDPE) or cellulose acetate. Other suitable materials include polyhydroxyalkanoate (PHA) fibres. The Aerosol-cooling element is between the rod of aerosol-generating substrate and the mouthpiece or blocker element.

In embodiments, the blocker element or mouthpiece element is preferably located at the downstream end or mouth end of the inhaler article. In embodiments, the mouthpiece element is a blocker element. In embodiments the blocker element is a filter. Where the blocker element is a filter, the filter filters the aerosol that is generated from the aerosol-generating substrate in the inhaler article to prevent large particles form entering the mouth of the user. The blocker may be formed of a segment of a fibrous filtration material.

In embodiments, the upstream element, the rod of aerosol-generating substrate, the aerosol-cooling element and the blocker element are held together by an outer wrapper circumscribing the upstream element, the aerosol-cooling element and the blocker element. The outer wrapper may be a hydrophobic paper. The outer wrapper may be paper. A hydrophobic wrapper may be a paper layer comprising PVOH (polyvinyl alcohol) or silicon. The PVOH may be applied to the paper layer as a surface coating, or the paper layer may comprise a surface treatment comprising PVOH or silicon.

In embodiments, the holder comprises a cavity for receiving the inhaler article and a heater. For example, the heater is an induction heater. The induction heater includes an induction source for generating an alternating magnetic field in the cavity which receives the inhaler article. The field is used to induce at least one of heat generating eddy currents or hysteresis losses in a susceptor which is located in thermal proximity or direct physical contact with the aerosol-generating substrate in the rod of aerosol-generating substrate. When a magnetic field is generated, the susceptor is heated. The susceptor heats the aerosol-generating substrate which releases aerosol and active ingredient into an airflow through the inhaler article and the holder to the user. In embodiments, the heater is an induction coil, wherein the induction coil is arranged around at least a portion of the receiving cavity.

The present disclosure provides multiple inhaler articles as described above, wherein the inhaler articles are packed into a pack. The present disclosure provides a pack containing a plurality of inhaler articles. For example, multiple inhaler articles are packed in a pack so that end surfaces of the plurality of inhaler articles are visible when the pack is opened. That is, the multiple inhaler articles are packed with and end surface “up” so that the end surfaces are visible at the top part of the pack when the pack is opened. In embodiments, the inhaler articles are packed into the pack in an “unused” state. When the pack is first opened, the inhaler articles are packed into the pack in an “unused” state. When the pack is first opened and the inhaler articles are packed into the pack in the “unused” state, all of the inhaler articles are packed in the pack in the same orientation. That is, when the pack is assembled, the multiple inhaler articles are oriented in the pack in the same way. When the pack is new, all of the inhaler articles are packed into the pack with the same end surface facing “up”. When the pack is new, all of the inhaler articles can be packed with the upstream element end surface facing up. When the pack is new, all of the inhaler articles can be packed with colored indicia visible when the pack is opened. When the pack is new, all of the inhaler articles can be packed with the downstream element end surface facing up. The orientation

In embodiments, an end surface of at least one inhaler article has colored indicia. In embodiments the upstream end surface of at least one inhaler article has colored indicia. In embodiments, multiple inhaler articles are packed into a pack in so that the upstream end surface of all of the inhaler articles in the pack are visible when the pack is opened. In embodiments, when the pack is first opened and all of the inhaler articles contained in the pack are unused, colored indicia on the end surface of the upstream element are visible. In embodiments, when the pack is first opened and all of the inhaler articles contained in the pack are unused, colored indicia on the end surface of the downstream element or blocker element are visible. When the pack is first opened, all of the unused inhaler articles are in the pack in an unused orientation. In an embodiment, when the pack is first opened, all of the unused inhaler articles are in the pack with an end surface having colored indicia visible. In an embodiment, when a pack is first opened, all of the unused inhaler articles are in the pack with the upstream end surface having colored indicia are visible.

A method includes the steps of removing an inhaler article from a pack, inserting the inhaler article into a holder to form an inhaler system, using the inhaler system to provide a compound to the lungs of a user, removing the used inhaler article from the holder and replacing the used inhaler article into the pack wherein the replaced used inhaler article is replaced in the pack in an orientation opposite of the unused orientation. The orientation opposite of the unused orientation is the “used orientation”. In embodiments, a pack contains a plurality of inhaler articles in an unused orientation. In embodiments, colored indicia are visible on the end surfaces of inhaler articles packed in a pack in the unused orientation. In embodiments, colored indicia are visible on the end surfaces of inhaler articles packed in a pack in the used orientation. In embodiments, a plurality of inhaler articles are arranged in the pack in a mixture of an unused orientation and a used orientation. In embodiments, in the unused orientation, colored indicia are visible (are facing “up”) on the end surface of the upstream element of an inhaler article in a pack. In embodiments, in the used orientation, colored indicia are not visible (are facing “down”) when a used inhaler article is replaced in a pack in an opposite orientation compared with the unused orientation of the inhaler article in the pack. In embodiments, a plurality of inhaler articles are arranged in a pack in a mixture of “up” and “down” orientation, so that colored indicia are visible on the end surface of inhaler articles arranged in the “up” orientation and no colored indicia are visible on the end surface of the inhaler articles arranged in the “down” orientation.

A method includes the step of scanning the end surfaces of multiple inhaler articles packed in a pack in a mixture of “up” or “unused” and “down” or “used” orientation and removing an inhaler article in the “up” or “unused” orientation for use in an inhaler system.

As used herein, the singular forms “a”, “an”, and “the” also encompass embodiments having plural referents, unless the content clearly dictates otherwise.

As used herein, “or” is generally employed in its sense including “and/or” unless the content clearly dictates otherwise. The term “and/or” means one or all of the listed elements or a combination of any two or more of the listed elements.

As used herein, “have”, “having”, “include”, “including”, “comprise”, “comprising” or the like are used in their open-ended sense, and generally mean “including, but not limited to”. It will be understood that “consisting essentially of”, “consisting of”, and the like are subsumed in “comprising,” and the like.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

As used herein, “providing”, in the context of providing an apparatus or system, means manufacturing the apparatus or system, purchasing the apparatus or system, or otherwise obtaining the apparatus or system.

As used herein, “or” is generally employed in its sense including “and/or” unless the content clearly dictates otherwise. The term “and/or” means one or all of the listed elements or a combination of any two or more of the listed elements.

Any direction referred to herein such as “top”, “bottom”, “left”, “right”, upper”, “lower”, and other directions or orientations are described herein for clarity and brevity but are not intended to be limiting of an actual device or system. Devices and systems described herein may be used in a number of directions and orientations.

As used herein, “downstream” and “proximal” mean the mouthpiece end of the inhaler article. “Downstream” and “proximal” mean the end of the tubular inhaler article intended to be contacted by the mouth of a user. “Upstream” and “distal” mean the opposite end of the inhaler article. “Upstream” and “distal” mean the end of the tubular inhaler article intended to be inserted into the holder.

The term “nicotine” refers to nicotine and nicotine derivatives such as free-base nicotine, nicotine salts and the like.

As used herein the term “closed” means sufficiently closed to retain a capsule inside the inhaler article before and after use. “Closed” also means sufficiently closed to reduce loss of pharmaceutically active powder from the inhaler article before or after use. The inhaler article may be less closed after use than before use, but may still be considered closed if the function of reducing loss of the contents of the inhaler article is provided.

As used herein the term “use” means the steps of inserting an inhaler article into a holder, initiating an airflow through the holder and the inhaler article, and delivering of active ingredient to the lungs of a user. “Use” may optionally include the additional step of removing the inhaler article from the holder.

As used herein the term “longitudinal” refers to the direction corresponding to the main longitudinal axis of the inhaler article, which extends between the upstream and downstream ends of the inhaler article.

As used herein, the term “rod” denotes a generally cylindrical element of substantially circular, oval or elliptical cross-section.

As used herein, the term “color” means any color detectable by a user. “Color” includes white.

As used herein, the term “indicia” means a color or one or more symbols that are visible and recognizable by a user. A symbol may include a cut or a flap. Indicia may convey information to the user. This information may relate to the origin of the product, flavor, strength, other information or a combination thereof.

As used herein, the term “colored indicia” means a color or one or more symbols that are visible to a user. Where indicia is colored, it has a color that is different from the color of the inhaler article. That is, if the inhaler article is white, the colored indicia may be a color other than white. A white indicia on a white inhaler article would not be visible to a user. To be visible and recognizable, the colored indicia must be a different color from the inherent color of the inhaler article. Therefore, “colored indicia” is any use of a color which is visible to a user. “Colored indicia” may be a color on a surface of an inhaler article. For example, “colored indicia” may mean that an end face of an inhaler article is a color that is different from the color of the rest of the inhaler article. Or, “colored indicia” may be a shape that is a different color from the color of the rest of the inhaler article. For example “colored indicia” may be a square of color that is different form the color of the rest of the inhaler article. “Colored indicia” may be a circle of color that is different form the color of the rest of the inhaler article. “Colored indicia” may be a rectangle of color that is different form the color of the rest of the inhaler article. “Colored indicia” may be any shape. “Colored indicia” may be in the form of a symbol. That is, a symbol may be a color that is different from the color of the inhaler article. “Colored indicia” and “color indicia” are terms that are used interchangeably.

As used herein, the term “symbol” means a mark or character, a shape, a letter, a picture, a pictograph, a hieroglyph, an emoji or other visual element. As used herein, “symbol” may represent or indicate an idea, including but not limited to the origin, strength, flavor or nature of a product. A symbol may be one or more symbols.

As used herein, the term “pack” means a container containing multiple inhaler articles.

The invention is defined in the claims. However, below there is provided a non-exhaustive list of non-limiting examples. Any one or more of the features of these examples may be combined with any one or more features of another example, embodiment, or aspect described herein.

Example Ex1: An inhaler article for insertion into a holder to form an inhaler system for providing a compound to the lungs of a user, the inhaler article comprising a tubular body extending along a longitudinal axis from a downstream mouthpiece end to an upstream end; the upstream end comprising an upstream element; the upstream element comprising an upstream end surface; wherein the upstream end surface comprises indicia.

Example Ex2: The inhaler article of Example Ex1 wherein the indicia comprises colored indicia.

Example Ex3. The inhaler article of Example Ex1 or Example Ex2 wherein the indicia comprises at least one symbol.

Example Ex4. The inhaler article of any one of the preceding Examples wherein the tubular body comprises, in linear sequential arrangement, from the upstream end to the downstream mouthpiece end: an upstream element; a capsule cavity containing a capsule; the capsule containing dry powder active ingredient; a blocker element.

Example Ex5. The inhaler article Example Ex4 wherein the upstream element comprises a resilient element.

Example Ex6. The inhaler article of Example Ex5 wherein the resilient element comprises an applied resilient element.

Example Ex7. The inhaler article of Example Ex6 wherein the applied resilient element is affixed to the upstream end of the tubular body.

Example Ex8. The inhaler article of any one of Examples Ex5-Ex7 wherein the resilient element comprises indicia.

Example Ex9. The inhaler article of any one of Examples Ex5-Ex8 wherein the resilient element comprises cuts which form flaps.

Example Ex10. The inhaler article of Example Ex9 wherein the resilient element comprises at least 2 diametric cuts to form at least 4 flaps.

Example Ex11. The inhaler article of Example Ex 9 wherein the resilient element comprises at least 3 diametric cuts to form at least 6 flaps.

Example Ex12. The inhaler article of any one of Examples Ex 9-Ex11 wherein the cuts extend a range from about 654% to about 95% of a diameter of the resilient element.

Example Ex13. The inhaler article of any of Examples Ex5-Ex8 wherein the resilient element comprises a central aperture.

Example Ex14. The inhaler article according to Example Ex13 wherein the central aperture has a diameter comprising less than 30% of a diameter of the resilient element.

Example Ex15. The inhaler article of any one of the Examples Ex5 to Ex14 wherein the resilient element comprises silicon, latex, rubber, plastic, paper, aluminium foil, paper tape, laminated layered PLA on a paper layer, or cardboard.

Example Ex16. The inhaler article of any one of Examples Ex5 to Ex15 wherein the resilient element comprises silicon, latex, rubber, or a combination thereof.

Example Ex17. The inhaler article of any one of Examples Ex1 to Ex16 wherein the blocker element comprises a filter.

Example Ex18.The inhaler article according to any of the preceding Examples further comprising a holder, wherein the holder comprises a piercing element.

Example Ex19. The inhaler article according to any one of Examples Ex1-Ex3 wherein the tubular body comprises, in linear sequential arrangement, from the upstream end to the downstream mouthpiece end: an upstream element, a rod of aerosol-generating substrate located downstream of the upstream element, an aerosol-cooling element located downstream of the support element, a blocker element located downstream of the aerosol-cooling element, and an outer wrapper circumscribing the upstream element, the aerosol-cooling element and the blocker element.

Example Ex20. The inhaler article according to Example Ex19, wherein the aerosol-generating substrate comprises tobacco.

Example Ex21. The inhaler article according to Examples Ex19 or Ex20, further comprising a holder, wherein the holder comprises a heater.

Example Ex22. The inhaler article according to Example Ex21, wherein the heater comprises an induction heater.

Example Ex23. The inhaler article according to any one of the preceding Examples further comprising a pack, the pack containing a plurality of inhaler articles.

Example Ex 24. The inhaler article according to Example 23, wherein at least one upstream end surface comprising indicia of at least one inhaler article contained in the pack is visible when the pack is open.

Example Ex25. A pack according to Example Ex23 or Example Ex24 comprising a plurality of inhaler articles according to any one of the preceding Examples arranged in the pack in an unused orientation.

Example Ex26. A pack according to Example Ex23 or Example Ex24 wherein the plurality of inhaler articles are arranged in the pack in a mixture of an unused orientation and a used orientation.

Examples will now be further described with reference to the figures in which:

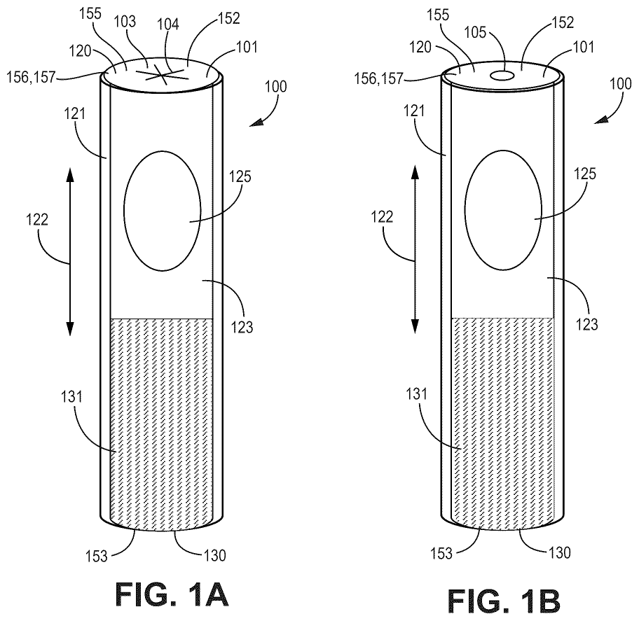

FIG. 1A and FIG. 1B show embodiments of inhaler articles having resilient elements. FIG. 1A illustrates an inhaler article having a resilient element having flaps on the upstream end of the inhaler article. FIG. 1B illustrates an inhaler article having a resilient element having a central aperture on the upstream end of the inhaler article.

FIG. 2 shows an inhaler article of the present disclosure inserted into a holder forming an inhaler system.

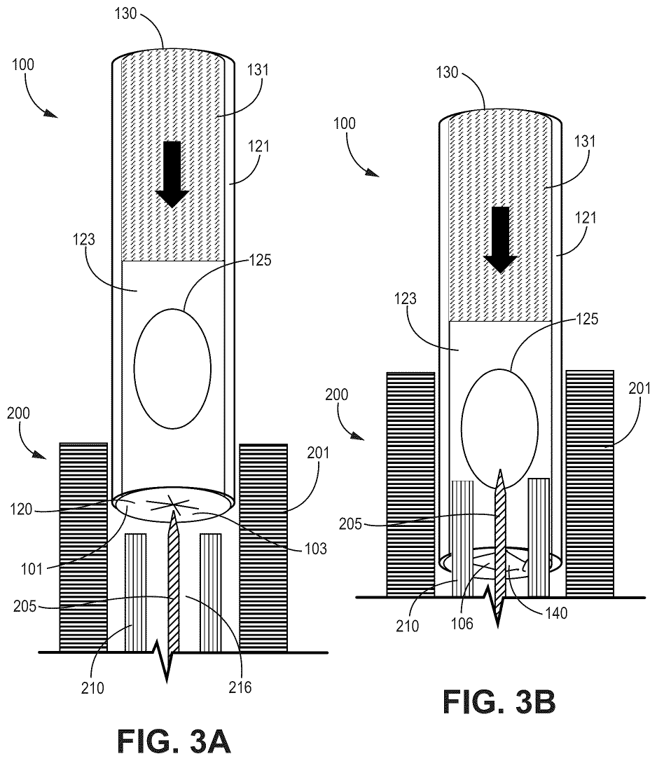

FIG. 3A and FIG. 3B illustrate the insertion of an inhaler article of the present disclosure into a holder. FIG. 3A illustrates an inhaler article which has a resilient element at the upstream end, pre-cut to form flaps. FIG. 3B illustrates the inhaler article having a resilient element at the upstream end, pre-cut to form flaps, inserted into a holder, where the flaps have been opened by the holder.

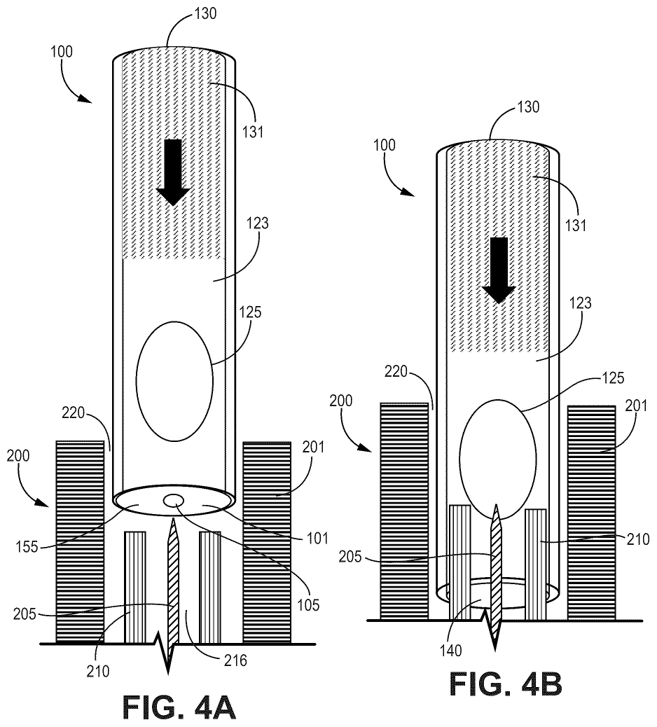

FIG. 4A and FIG. 4B illustrate the insertion of an inhaler article of the present disclosure into a holder. FIG. 4A illustrates an inhaler article which has a resilient element at the upstream end, the resilient element having a central aperture. FIG. 4B illustrates the inhaler article having a resilient element at the upstream end, having a central aperture, inserted into a holder, where the central aperture has been opened by the holder.

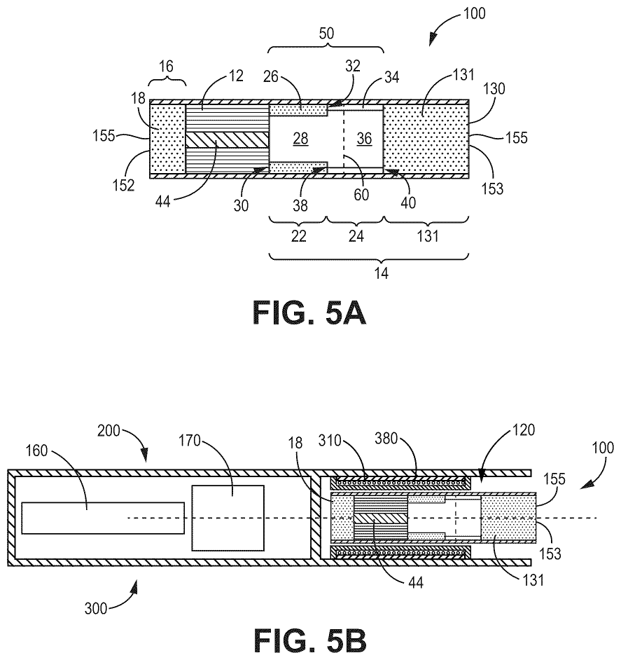

FIG. 5A is an embodiment of the inhaler article of the present disclosure for use in a holder which has a heater. FIG. 5B is an illustration of an embodiment of an inhaler article of the present disclosure engaged with an embodiment of a holder, forming an inhaler system.

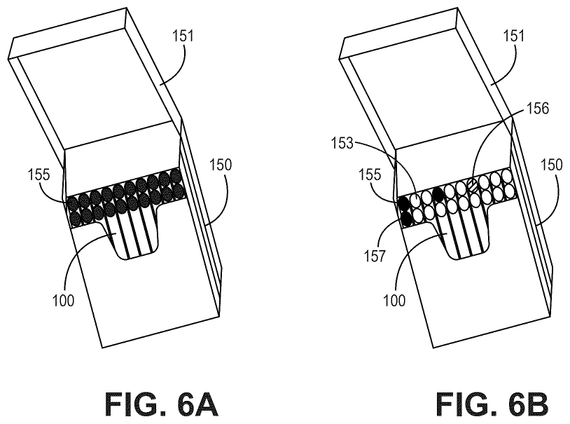

FIG. 6A and FIG. 6B show embodiments of packs containing a plurality of inhaler article of the present disclosure. FIG. 6A shows a pack containing a plurality of inhaler articles in a pack with no indicia visible. FIG. 6B shows a pack containing a plurality of inhaler articles with a few of the inhaler articles having visible colored indicia.

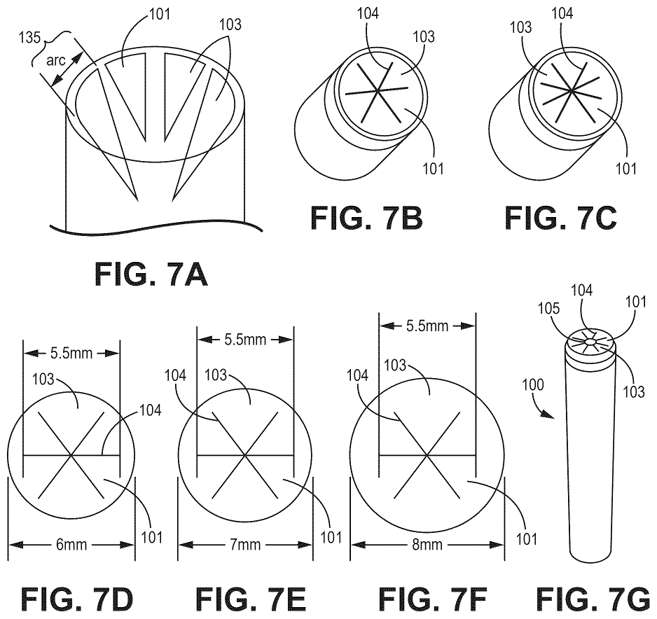

FIG. 7A, FIG. 7B, FIG. 7C, FIG. 7D, FIG. 7E, FIG. 7F and FIG. 7G illustrate embodiments of the inhaler article of the present disclosure having a resilient element at the upstream end, pre-cut to form flaps. FIG. 7A illustrates the flaps folding back to form an internal opening. FIG. 7B illustrates an embodiment of the inhaler article of the present disclosure having a resilient element at the upstream end, pre-cut to form flaps, having 6 radial cuts or 3 diametric cuts, to form 6 flaps. FIG. 7C illustrates an embodiment of the inhaler article of the present disclosure having a resilient element at the upstream end, pre-cut to form flaps, having 8 radial cuts or 4 diametric cuts, to form 8 flaps. FIG. 7D illustrates an embodiment of the inhaler article of the present disclosure having a resilient element at the upstream end, pre-cut to form flaps, having 6 radial cuts or 3 diametric cuts, to form 6 flaps illustrating, in an embodiment, a ratio of the length of the cuts in relation to the diameter of the inhaler article. FIG. 7E illustrates an embodiment of the inhaler article of the present disclosure having a resilient element at the upstream end, pre-cut to form flaps, having 6 radial cuts or 3 diametric cuts, to form 6 flaps illustrating, in an embodiment, a ratio of the length of the cuts in relation to the diameter of the inhaler article. FIG. 7F illustrates an embodiment of the inhaler article of the present disclosure having a resilient element at the upstream end, pre-cut to form flaps, having 6 radial cuts or 3 diametric cuts, to form 6 flaps illustrating, in an embodiment, a ratio of the length of the cuts in relation to the diameter of the inhaler article. FIG. 7G is an illustration of an embodiment of the inhaler article having a central aperture and flaps and having color as indicia.

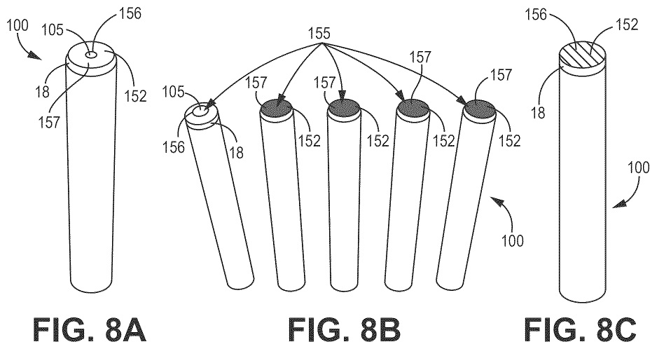

FIG. 8A, FIG. 8B, and FIG. 8C are illustrations of embodiments of inhaler articles of the present disclosure having colored indicia at the upstream end surface of the inhaler articles. FIG. 8A is an illustration of a resilient element at the upstream end, where the resilient element has a central aperture and where the resilient element has color. FIG. 8B is an illustration of the several inhaler articles having different color indicia. FIG. 8C is an illustration of an inhaler article having symbols as indicia on the upstream end face of the inhaler article.

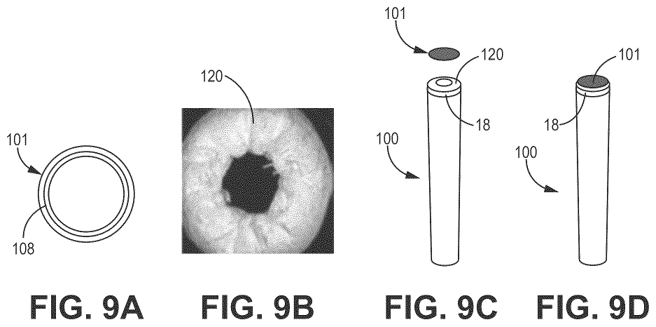

FIG. 9A, FIG. 9B FIG. 9C and FIG. 9D are illustrations of embodiments of the inhaler article 100 of the present disclosure. FIG. 9A is an illustration of the resilient element 101, having an applied ring of glue 108, prior to affixing the resilient element 101 to the upstream end 120 of the inhaler article 100. FIG. 9B is a photograph of an embodiment of the upstream end 120 of the inhaler article 100 before the resilient element 101 is affixed. FIG. 9C is an illustration of the application of a resilient element 101 to the upstream end 120 of the inhaler article 100. FIG. 9D is an illustration of the inhaler article 100 after the resilient element has been affixed to the upstream end 120 of the inhaler article 100.

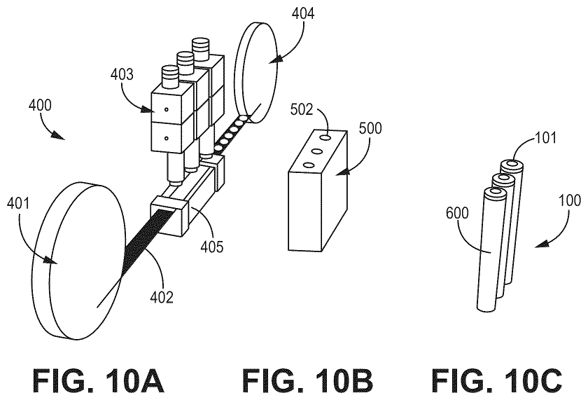

FIG. 10A, FIG. 10B and FIG. 10C illustrate manufacturing equipment for manufacturing embodiments of the inhaler article having a resilient element affixed to the upstream end of the inhaler article. FIG. 10A illustrates a ribbon cutter to cut diskettes of resilient material to create resilient elements. FIG. 10B shows a stage to provide a ring of glue in the manufacturing process. FIG. 10C shows a holder containing three inhaler articles, which will be presented to the resilient elements having an applied glue ring, during the manufacturing process.

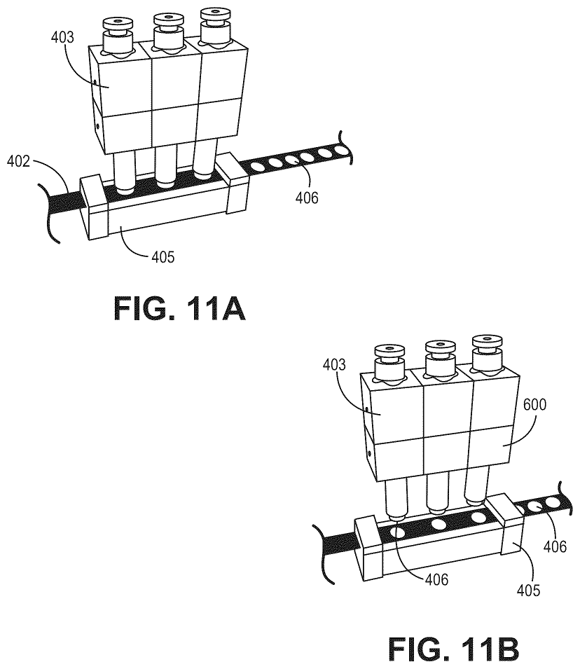

FIG. 11A and FIG. 11B illustrate manufacturing equipment for manufacturing embodiments of the inhaler article having a resilient element affixed to the upstream end of the inhaler article. FIG. 11A shows the ribbon cutter before resilient elements of resilient material have been cut from a ribbon of resilient material. FIG. 11B shows the ribbon cutter after disks of resilient material have been cut from a ribbon of resilient material.

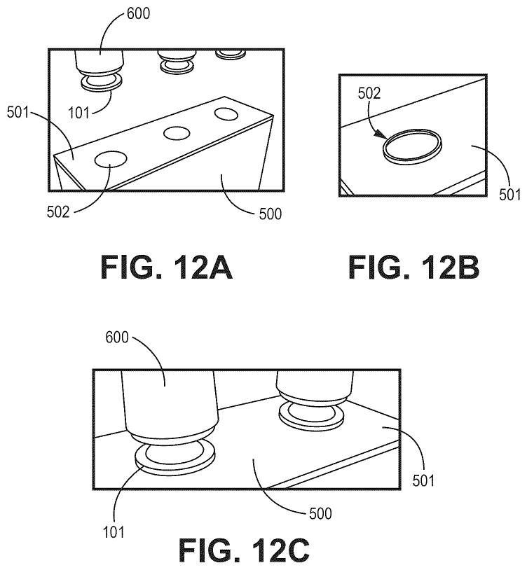

FIG. 12A, FIG. 12B, and FIG. 12C illustrate manufacturing equipment and methods for affixing the resilient element to the inhaler article in embodiments of the inhaler article having a resilient element affixed to the upstream end of the inhaler article. FIG. 12A illustrates a step of presenting the resilient element to the gluing station. FIG. 12B illustrates the step of loading the glue ring. FIG. 12C illustrates the step of applying glue to the resilient element.

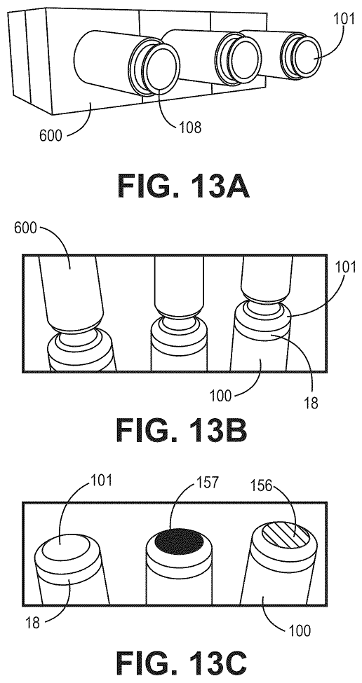

FIG. 13A, FIG. 13B, and FIG. 13C illustrate manufacturing equipment for manufacturing embodiments of the inhaler article having a resilient element affixed to the upstream end of the inhaler article. FIG. 13A illustrates a perspective view of the resilient element having an applied ring of glue, after the manufacturing step of FIG. 13C. FIG. 13B illustrates the presentation of the inhaler article to the glue-side of the resilient element having an applied ring of glue. FIG. 13C illustrates the inhaler article having a resilient element affixed to the upstream end of the inhaler article.

The schematic drawings are not necessarily to scale and are presented for purposes of illustration and not limitation. The drawings depict one or more aspects described in this disclosure. However, it will be understood that other aspects not depicted in the drawing fall within the scope and spirit of this disclosure.

FIG. 1A illustrates an inhaler article 100 having an upstream end 120 (also a distal end), a downstream end 130 (also a proximal end or a mouthpiece end), a tubular body 121 extending along a longitudinal axis 122 from the downstream end 130 to an upstream end 120. The upstream end 120 has an upstream end face 152. The downstream end 130 has a downstream end face 153. The upstream end face 152 may have colored indicia 155. The downstream end face 153 may have colored indicia 155. Both the upstream end face 152 and the downstream end face 153 may have colored indicia. Indicia 155 may be colored indicia 157. Indicia 155 may be one or more symbols 156. Indicia 155 may be both visible color 157 and one or more symbols 156. Symbol indicia 156 may be colored symbol indicia 156. FIG. 1A shows an embodiment of the inhaler article which has a resilient element 101 at the upstream end 120 which is cut 104 to form flaps 103. FIG. 1A shows the cuts 104 and flaps 103 formed by the cuts 104. The resilient element may have indicia 155. The resilient element may be indicia 155. For example, the resilient element may be colored, and may provide color indicia 157. The resilient element may be a different color from the color of the rest of the inhaler article. For example, the colored indicia 157 may be blue. The colored indicia 157 may be red. The colored indicia 157 may be orange. The colored indicia 157 may be black. The colored indicia 157 may be green. The colored indicia 157 may be yellow. The colored indicia 157 may be a design having a color different from the background color of the inhaler article 100. Or, the resilient element may have at least one symbol indicia 156. For example, flaps 103 are symbol indicia 156. Indicia may indicate origin of the inhaler article. Indicia may indicate flavor of the inhaler article. Indicia may indicate the nature of the powdered active ingredient in the inhaler article. Indicia may indicate strength of the active ingredient in the inhaler article. Indicia may indicate whether the inhaler article has been used. Indicia may indicate that the inhaler article is suitable for use with the holder. Indicia may provide information to the user related to the inhaler article.

FIG. 1A and FIG. 1B also show the capsule cavity 123 containing a capsule 125. FIG. 1B shows an embodiment of the inhaler article which has a resilient element 101 at the upstream end 120, the resilient element having a central aperture 105. FIG. 1A and FIG. 1B also show that the inhaler article 100 may have a blocker 131 located near the downstream end 130 of the inhaler article 100. The central aperture 105 is a symbol indicia 156.

The tubular body 121 may be made of a carton or wrapping paper rolled in a tube form. The resilient element 101 has an opening, formed, as shown in FIG. 1A, by cuts 104 forming flaps 103. These cut flaps 103 provide an opening in the resilient element 101. In FIG. 1B, the central aperture 105 is shown. These openings provide access for a piercing pin 205 to be able to reach and perforate the capsule 125 when the inhaler article 100 is placed in the holder 200 and the capsule 125 is activated. At the same time, these openings are smaller than the diameter of the capsule 125, to prevent the capsule 125 from falling out of the inhaler article 100 before, during or after use. FIG. 1A and FIG. 1B also show a blocker element 131.

FIG. 2 shows an inhaler article 100 of the present disclosure inserted into a holder 200 forming an inhaler system 300. The inhaler article 100 has a capsule 125, a capsule cavity 123 and a tubular body 121. The holder 200 has a housing 201 defining a housing cavity 216. The housing cavity 216 has an open end 220 and a piercing end 221. When the inhaler article 100 is introduced into the holder 200, into the housing cavity 216, the inhaler article 100 pushes against the moveable cap 202 at the distal end of the housing cavity 216. The moveable cap 202 has prongs 210. These prongs 210 may be part of the moveable cap or may separate from the moveable cap. The prongs 210 are located in the housing cavity 216. Prongs 210 are structured to insert into the tubular body 121 of the inhaler article 100 when the inhaler article 100 is inserted into the holder 200. The moveable cap 202 moves down relative to the housing 201 as shown by the arrow. When the inhaler article and the moveable cap 202 move down in relation to the housing 201, the piercing pin 205 extends through the resilient element (not shown in FIG. 2 but see FIG. 3A and FIG. 3B) and pierces the capsule 125.

There is an air inlet 206 through the housing 201 which allows air to enter the inhaler system 300. Air flows in an air flow path 301 into the inhaler system 300 through the air inlet 206, through the airflow passage 208 through the moveable cap 202, into the capsule cavity 123 of the inhaler article 100. Because the airflow passage 208 is tangential to the longitudinal axis 122 of the inhaler article 100 (and the capsule cavity 123), air flowing through the capsule cavity 123 flows in a swirling airflow path 302. In embodiments, there is one airflow passage 208. In embodiments, there are two airflow passages 208. In embodiments, there are more than two airflow passages 208.

When using such an article, the user will remove the inhaler article 100 from its packaging, which might include a pack 150 as shown in FIG. 6. The user will insert the inhaler article 100 into the holder 200 to form an inhaler system 300. When inserting the inhaler article 100 into the holder 200, the user presses down on the inhaler article which moves the moveable cap 202 in relation to the piercing pin 205, and moves the capsule 125, contained in the capsule cavity 123 of the inhaler article, to contact the piercing pin 205, piercing the capsule 125. Once the capsule 125 has been pierced, the moveable cap 202 retracts, by the action of a spring 212, for example. The movement of the moveable cap is indicated by arrow 215.

After the capsule 125 is pierced, the inhaler article moves away from the capsule 125 as shown in FIG. 2. air flows into the system via the air inlet 206, through the airflow passages 208, through the capsule cavity 123, through the blocker element 131 and out of the system to a user via the downstream end 130 of the inhaler article 100 to the mouth of a user. Powder released from the pierced capsule 125 is entrained into the airflow path 302 and powder is delivered to the downstream end 130 (the mouthpiece end) of the inhaler article 100 and is inhaled by the user. This airflow is initiated by suction provided by the user at the mouthpiece end, the downstream end 130 of the inhaler article 100. In addition, the swirling airflow path 302 provides agitating or swirling airflow which agitates the capsule 125 inside the capsule cavity 123, and improves release of powder from the capsule 125 during use.

It is desirable to be able to create an effective air flow through the inhaler system 300 to entrain an appropriate amount of powder in the airflow so as to provide an appropriate dose of powder to a user. The powder is released from the capsule 125 through a hole introduced into the capsule by the piercing pin 205. This piercing pin 205 also passes through the upstream end 120 of the inhaler article. If the upstream end 120 of the inhaler article 100 is closed, the piercing pin 205 introduces a hole in the upstream end 120 of the inhaler article 100. The hole made by the piercing pin 205 in the upstream end 120 of an inhaler article is generally related to the size of the piercing pin 205. In general, a hole made by a piercing pin 205 is not large enough to enable sufficient airflow through the inhaler system 300 to provide an appropriate dose of powder to the user. That is, a small hole in the upstream end 120 of the inhaler article 100 in an inhaler system 300 introduces significant resistance to draw (RTD) into the system and creates a system that does not have sufficient airflow to provide an appropriate dose of powder released from a capsule 125 to a user. One solution to this RTD challenge is to provide an inhaler article 100 having an upstream end 120 that is open when it is inserted into the holder.

After the inhaler system 300 has been used, the user can withdraw the inhaler article 100 from the holder 200. After the inhaler article 100 is withdrawn from the holder, the resilient element 101 reverts back to its pre-use state, or nearly its pre-use state. After the inhaler article 100 has been used and has been withdrawn from the holder, the inhaler article 100 can be replaced in the packaging or pack 150. As shown below in FIG. 6A, inhaler articles may be packed into a pack with colored indicia 153 on the upstream end of the articles 100 facing “up” or visible to the consumer. The inhaler article 100 can be inverted when it is replaced in the pack so that colored indicia, which may be color or at least one symbol provided by the resilient element, is not visible. That way, the consumer can tell at a glance which of the articles in the pack are used. Or, inhaler articles may be packed into a pack with colored indicia on the upstream end of the articles facing “down”, not visible to the consumer upon opening the pack. The inhaler article 100 can be inverted when it is replaced in the pack so that colored indicia, which may be color or at least one symbol provided by the resilient element, is visible. For example, inhaler articles may be provided in a pack with the resilient element 101 oriented “down” so that the resilient element is not visible to the user when the user opens the lid 151 of a pack 150 containing multiple unused inhaler articles 100. After use, when the user replaces the inhaler article in the pack, the user may invert the used inhaler article so that the resilient element of the used inhaler article is “up” and visible to the user. This colored indicia, visible to the user when the used inhaler article 100 is replaced in the pack, indicates to the user that the inhaler article showing colored indicia is used. The user can then distinguish between a used and an unused inhaler article in the pack. The user can choose an unused inhaler article for the next dose, and can repeat the process described above. Or, the inhaler article 100 can be inverted when it is replaced in the pack so that colored indicia, which may be color or at least one symbol provided by the resilient element, is not visible. For example, inhaler articles may be provided in a pack with the resilient element 101 oriented “up” so that the resilient element is visible to the user when the user opens the lid 151 of a pack 150 containing multiple unused inhaler articles 100. After use, when the user replaces the inhaler article in the pack, the user may invert the used inhaler article so that the resilient element of the used inhaler article is “down” and not visible to the user. The lack of colored indicia in this inverted inhaler article when the used inhaler article 100 is replaced in the pack, indicates to the user that the inhaler article not showing indicia is a used inhaler article. The user can then distinguish between a used and an unused inhaler article in the pack. The user can choose an unused inhaler article for the next dose, and can repeat the process described above. In addition, the presence of colored indicia 155 encourages the user to replace used inhaler articles in the pack instead of disposing the used inhaler article into the environment. The presence of colored indicia provides information to the user. The presence of colored indicia also reduces pollution.

It is desirable to ensure that the capsule 125, containing active ingredient is retained in the inhaler article before and after insertion of the inhaler article 100 into the holder 200. In addition, it is desirable to prevent powder from spilling from the inhaler article 100 before and after insertion of the inhaler article 100 into the holder 200. It is desirable to prevent the capsule powder from falling out of or spilling from the inhaler article 100 before and after use. Providing an inhaler article 100 having an upstream end 120 that is open may not retain the capsule 125 inside the capsule cavity 123 of the inhaler article 100. The present disclosure provides solutions that address both the RTD challenge and the retention challenge, and also provide information to the user and reduce pollution. These solutions are provided by resilient elements at the upstream end of the inhaler article, which can be closed (or relatively closed) to retain the capsule and powder before and after insertion into a holder 200 to form an inhaler system 300, and opened during use, during insertion into a holder 200, to provide appropriate airflow and an appropriate dose of powder to a user of the inhaler system 300, and which also comprise colored indicia which provides information to the user and also may reduce pollution.

As shown in FIG. 2, when inserting the inhaler article into the holder the user simultaneously moves the moveable cap 202, perforates the capsule 125, and inserts the insertion portion of the moveable cap 202 into the inhaler article to provide an airflow aperture 140 at the upstream end 120 of the inhaler article 100 which provides an appropriate RTD for the proper operation of the inhaler system 300. That is, the hole at the upstream end 120 of the inhaler article 100 is large enough for the inhaler system to function, when the insertion portion of the moveable cap is inserted into the inhaler article, and the resilient element 101 is moved or stretched open.

In addition, when the inhaler article 100 is removed from the holder 200, the resilient element reverts to its pre-insertion state (partially or fully). When the resilient element 101 reverts to its pre-insertion state, the capsule 125 and the powder are contained inside the inhaler article 100. However, the resilient element 101 comprising colored indicia 155 remains, and can provide information to the user and also can reduce pollution.

FIG. 3A and FIG. 3B illustrate the insertion of an inhaler article 100 having a resilient element 101 having flaps 103 into the holder 200. The inhaler article 100 is inserted into the housing cavity 216 of the holder 200. The arrows in FIG. 3A and FIG. 3B illustrate the insertion of the inhaler article 100 into the holder 200. FIG. 3A illustrates an inhaler article 100 which has a resilient element 101 at the upstream end 120, which has flaps 103. FIG. 3B illustrates the inhaler article 100 having a resilient element 101 at the upstream end 120, having flaps 103, inserted into a holder 200, where the flaps 103 have been opened 106 by the prongs of the moveable cap 210 of the holder 200. When the flaps 103 are opened, an airflow aperture 140 is created. This airflow aperture 140 creates a large area for swirling airflow to flow through the inhaler system 300. Flaps 103, cut into the resilient element 101, are indicia 155. That is, flaps 103 are symbols in the form of flap-shapes in the resilient element 101.

After the inhaler system 300 has been used, the user can withdraw the inhaler article 100 from the holder 200. After the inhaler article 100 is withdrawn from the holder, and the prongs of the moveable cap 210 is removed from the inhaler article 100, the resilient element 101 reverts back to its pre-use state, or nearly its pre-use state. After use, the flaps 103 on the upstream end 120 of the inhaler article 100 revert back to a closed state, as shown in, for example, FIG. 1A.

FIG. 4A and FIG. 4B illustrate the insertion of an inhaler article 100 of the present disclosure into a holder 200 to form an inhaler system 300. The arrows in FIG. 4A and FIG. 4B illustrate the insertion of the inhaler article 100 into the housing cavity 216 of the holder 200. FIG. 4A illustrates an inhaler article 100 which has a resilient element 101 at the upstream end 120, the resilient element 101 having a central aperture 105. FIG. 4B illustrates the inhaler article 100 having a resilient element 101 at the upstream end 120, having a central aperture 105, inserted into a holder 200, where the central aperture 105 has been opened to form an airflow aperture 140. The central aperture 105 has been opened to form an airflow aperture 140 by inserting the prongs of the moveable cap 210 through the central aperture 105 of the resilient element 101 of the inhaler article 100. A central aperture 105, provided in the resilient element 101, is indicia 155. That is, a central aperture 105 is a symbol in the form of central aperture-shapes in the resilient element 101. In embodiments, the diameter of the central aperture 105 may be about 30% of the diameter of the resilient element. In embodiments, the diameter of the central aperture may be less than 30% of the diameter of the resilient element.

After the inhaler system 300 has been used, the user can withdraw the inhaler article 100 from the holder 200. After the inhaler article 100 is withdrawn from the holder, and prongs of the moveable cap 210 are removed from the inhaler article 100, the resilient element 101 reverts back to its pre-use state, or nearly its pre-use state. After use, the central aperture 105 of the resilient element 101 reverts back to a closed state, as shown in, for example FIG. 1B. When the inhaler article 100 is removed from the holder 200, the resilient element reverts to its pre-insertion state (partially or fully). When the resilient element 101 reverts to its pre-insertion state, the capsule 125 and the powder are contained inside the inhaler article 100. However, the resilient element 101 comprising indicia 155 remains, and can provide information to the user and also can reduce pollution.

FIG. 5A is an embodiment of the inhaler article of the present disclosure for use in a holder which has a heater. The inhaler article 100 shown in FIG. 5A comprises a rod 12 of aerosol-generating substrate 12 and a downstream section 14 at a location downstream of the rod 12 of aerosol-generating substrate. Further, the inhaler article 100 comprises an upstream section 16 at a location upstream of the rod 12 of aerosol-generating substrate. Thus, the inhaler article 100 extends from an upstream end face 152 and an upstream element 18 to a downstream or mouth end 130 having a downstream end face 153. The inhaler article has an overall length of about 45 millimetres. The downstream section 14 comprises a support element 22 located immediately downstream of the rod 12 of aerosol-generating substrate, the support element 22 being in longitudinal alignment with the rod 12. In the embodiment of FIG. 5A, the upstream element 18 abuts the upstream end of the rod 12 of aerosol-generating substrate. In addition, the downstream section 14 comprises an aerosol-cooling element 24 located immediately downstream of the support element 22, the aerosol-cooling element 24 being in longitudinal alignment with the rod 12 and the support element 22. In the embodiment of FIG. 5A, the upstream end of the aerosol-cooling element 24 abuts the downstream end of the support element 22. In this embodiment, the inhaler article has an upstream element 18.

The support element 22 and the aerosol-cooling element 24 together define an intermediate hollow section 50 of the inhaler article 100. The support element 22 comprises a first hollow tubular segment 26. The first hollow tubular segment 26 is provided in the form of a hollow cylindrical tube made of cellulose acetate. The first hollow tubular segment 26 defines an internal cavity 28 that extends all the way from an upstream end 30 of the first hollow tubular segment to a downstream end 32 of the first hollow tubular segment 26. The internal cavity 28 is substantially empty, and so substantially unrestricted airflow is enabled along the internal cavity 28. The aerosol-cooling element 24 comprises a second hollow tubular segment 34. The second hollow tubular segment 34 is provided in the form of a hollow cylindrical tube made of cellulose acetate. The second hollow tubular segment 34 defines an internal cavity 36 that extends all the way from an upstream end 38 of the second hollow tubular segment to a downstream end 40 of the second hollow tubular segment 34. The internal cavity 36 is substantially empty, and so substantially unrestricted airflow is enabled along the internal cavity 36. the downstream section 14 further comprises a blocker element 131 at a location downstream of the intermediate hollow section 50. In more detail, the blocker element 131 is positioned immediately downstream of the aerosol-cooling element 24. As shown in the drawing of FIG. 5A, an upstream end of the blocker element 131 abuts the downstream end 40 of the aerosol-cooling element 24. The blocker element 131 is provided in the form of a cylindrical plug of low-density cellulose acetate. The blocker element 131 can be a filter. The blocker element 131 has a length of about 12 millimetres and an external diameter of about 7.25 millimetres. The RTD of the mouthpiece element 131 is about 12 1 0 millimetres H2O. The rod 12 comprises an aerosol-generating substrate of one of the types described above. The rod 12 of aerosol-generating substrate has an external diameter of about 7.25 millimetres and a length of about 12 millimetres.

The inhaler article 100 further comprises an elongate susceptor 44 within the rod 12 of aerosol-generating substrate. In more detail, the susceptor 44 is arranged substantially longitudinally within the aerosol-generating substrate, such as to be approximately parallel to the longitudinal direction of the rod 12. The susceptor 44 is positioned in a radially central position within the rod and extends effectively along the longitudinal axis of the rod 12. The susceptor 44 extends all the way from an upstream end to a downstream end of the rod 12. In effect, the susceptor 44 has substantially the same length as the rod 12 of aerosol-generating substrate.

In the embodiment of FIG. 5A, the susceptor 44 is provided in the form of a strip and has a length of about 12 millimetres, a thickness of about 60 micrometres, and a width of about 4 millimetres. The upstream section 16 comprises an upstream element 18 located immediately upstream of the rod 12 of aerosol-generating substrate, the upstream element 18 being in longitudinal alignment with the rod 12. In the embodiment of FIG. 5A, the downstream end of the upstream element 18 abuts the upstream end of the rod 12 of aerosol-generating substrate. This advantageously prevents the susceptor 44 from being dislodged. Further, this ensures that the consumer cannot accidentally contact the heated susceptor 44 after use. The upstream element 18 is provided in the form of a cylindrical plug of cellulose acetate circumscribed by a stiff wrapper. The upstream element 18 has a length of about 5 millimetres.

FIG. 5B is an illustration of an embodiment of an inhaler article 100 of the present disclosure engaged with an embodiment of a holder 200, forming an inhaler system 300. As shown in FIG. 5B, the inhaler article 100 of FIG. 5A is inserted into a holder 200 having a cavity 120 to receive and accommodate the inhaler article 100. The holder 200 comprises a power supply 160, for example a lithium ion battery, and an electric circuitry 170 including a controller for controlling operation of the inhaler system 300, in particular for controlling the heating process. The holder 200 comprises an induction source including an induction coil 310 for generating an alternating, in particular high-frequency magnetic field. The induction coil 310 is a helical coil circumferentially surrounding the cylindrical receiving cavity 120. The induction coil 310 is formed from a wire 380 and has a plurality of turns, or windings, extending along its length. The wire 380 has a circular cross-section. In other embodiments, the wire may have a flat cross-sectional shape.

In use, the embodiment of FIG. 5A and FIG. 5B when the device is actuated, a high-frequency alternating current is passed through the induction coil 310. This causes the coil 310 to generate an alternating magnetic field within the cavity 120. As a consequence, the susceptor 44, located in the rod 12 of aerosol-generating substrate heats up due to at least one of eddy currents or hysteresis losses, depending on the magnetic and electric properties of the materials of the susceptor element 44. The susceptor 44 in turn heats the rod 12 of aerosol-generating substrate to a temperature sufficient to form an aerosol. The aerosol may be drawn downstream through the inhaler article 100 for inhalation by the user.

In embodiments, the upstream end face 152 of the inhaler article 100 comprises colored indicia 155. In embodiments, the downstream end face 153 of the inhaler article 100 comprises colored indicia. In embodiments both the upstream end face 152 and the downstream end face 153 of the inhaler article comprises colored indicia 155. In embodiments, the indicia 155 is color indicia 157. In embodiments, the indicia 155 is at least one symbol 156. In embodiments, the indicia is both color 157 and at least one symbol 156. In embodiments, the colored indicia 155 on the upstream end face 152 is a different color from the colored indicia 155 on the downstream end face 153 of the inhaler article 100.

In embodiments, the inhaler article comprises a tubular body, in linear sequential arrangement, from the upstream end to the downstream mouthpiece end: an upstream element, a rod of aerosol-generating substrate located downstream of the upstream element, an aerosol-cooling element located downstream of the rod of aerosol-generating substrate, a blocker element located downstream of the aerosol-cooling element, and an outer wrapper circumscribing the upstream element, the aerosol-cooling element and the blocker element.

FIG. 6A and FIG. 6B are illustrations of a plurality of inhaler articles 100 of the present disclosure packed into a pack 150. The pack 150 has a lid 151. When the lid 151 of the pack 150 is opened, end faces of the plurality of inhaler articles 100 are visible to the user. End faces that are visible when the lid 151 of the pack is opened are “up”. End faces that are not visible when the lid 151 of the pack 150 is opened, end faces that are inserted into the pack, these end faces are “down”. Visible end faces can be upstream end faces 152 or downstream end faces 153 depending on the orientation of the inhaler articles as they are packed into the pack 150. Initially, as inhaler articles are packed into a pack 150 during manufacturing, all of the inhaler articles are initially packed into a pack in the same orientation. That is, either the upstream end faces 152 or the downstream end faces 153 will be “up” and visible when the lid 151 of the pack 150 is opened.

FIG. 6A is an illustration showing inhaler articles 100 packed into a pack 150. In FIG. 6A, indicia 155 are visible when the lid 151 of the pack 150 is opened. That is, indicia 155 are oriented “up” in the pack. In this embodiment, inhaler articles have indicia 155 on upstream end faces 152 and no indicia on downstream end faces 153. As shown in FIG. 6A, upstream end faces 152 are up and downstream end faces 153 are down. Downstream end faces 153 do not have indicia 155. The opposing end face, the upstream end faces 152, are facing “up” and are visible with the lid of the pack 150 is opened. Therefore, as shown in FIG. 6A, indicia 155 are visible when the lid 151 of the pack 150 is opened. That is, in this illustration of FIG. 6A, the inhaler articles 100 are packed into the pack with the upstream ends inside the pack (the upstream end is “up”) and the downstream end faces 153 are at the bottom of the pack and are not visible when the lid 151 of the pack 150 is opened (the downstream end faces 153 are “up”). That is, as shown in FIG. 6A, the inhaler articles are packed into the pack 150 with the upstream ends up.

In embodiments, inhaler articles may be packed into a pack 150 with the upstream end faces 152 down and the downstream end faces 153 up. That is, in embodiments, inhaler articles may be packed into a pack so that the consumer sees downstream end faces 153 upon opening the pack. In embodiments, downstream end faces 153 have colored indicia 155.

FIG. 6B shows that some of the visible end faces of the plurality of inhaler articles 100 can have colored indicia 155 that are visible when the lid 151 of the pack 150 is opened. The indicia 155 can be colored indicia 157. For example, the colored indicia 157 may be blue. The colored indicia 157 may be red. The colored indicia 157 may be orange. The colored indicia 157 may be black. The colored indicia 157 may be green. The colored indicia 157 may be yellow. The colored indicia 157 may be a design having a color different from the background color of the inhaler article 100. Indicia can be symbol indicia 156. Indicia can be both color 157 and symbol 156 indicia. Indicia may be on a resilient element 101. In the embodiment shown in FIG. 6A and FIG. 6B indicia 155 is present on the upstream end face 152 of the inhaler articles 100, and no indicia 155 is present on the downstream end face 153 of the inhaler articles 100.

As illustrated in FIG. 6B, a pack 150 may contain some inhaler articles 100 in the pack 150 with the downstream end 153, without indicia, facing up. And, the same pack 150 may contain some inhaler articles 100 in the pack 150 with the upstream end 152, with indicia 155, facing up. That is, some of the inhaler articles 100 are inverted in the pack, as shown in FIG. 6B. In this way, indicia 155 on the upstream end faces 152 of these inverted inhaler articles are visible when the lid 151 of the pack 150 is opened. This can occur when a user removes an inhaler article from a new pack 150, as shown in FIG. 6A, where all of the inhaler articles 100 are packed into the pack 150 with visible indicia on the upstream end faces 152 facing up. The user then uses the inhaler article that has been removed from the pack 150. When the user replaces the used inhaler article into the pack 150, the user inverts the used inhaler article 100 and places the used inhaler article 100 into the pack with the downstream face 153 without indicia 155 facing “up”. That is, the user inverts the inhaler article when the user replaces a used inhaler article into the pack 150. As can be seen in FIG. 6B, some of the inhaler articles 100 have visible indicia 155 and some of the inhaler articles do not have visible indicia. When the user removes the next inhaler article from the pack, the user can see which inhaler article has been used, because the used inhaler articles are inverted in the pack, and indicia 155 on the upstream end face 152 of the unused inhaler articles 100 indicate to the user that the inhaler article 100 with visible indicia 155 is new. Inhaler articles that do not have visible indicia, inhaler articles which have been inverted when they are re-inserted into the pack, are used. This enables the user to easily distinguish between new and used inhaler articles 100 contained in the pack 150. Because the user is able to distinguish between new and used inhaler articles in the pack, the user is more likely to replace a used inhaler article in the pack and less likely to discard a used inhaler article into the environment.

FIG. 7A, FIG. 7B, FIG. 7C, FIG. 7D, FIG. 7E, FIG. 7F and FIG. 7G illustrate embodiments of the inhaler article of the present disclosure having a resilient element 101 at the upstream end, cut to form flaps 103. The number of cuts 104 and length of the cuts 104 define the geometry of the flaps 103 that are formed by the cuts 104. FIG. 7A illustrates the flaps 103 folding back to form an internal opening. FIG. 7A shows that the number of cuts 104, define the arc 135, the shape of the flap 103 where the flaps fold inward. The number of cuts (3 diametric cuts or 6 radial cuts as shown in FIG. 7B or 8 radial cuts or 4 diametric cuts as shown in FIG. 7C) changes the arc 135. Increasing the number of cuts reduces the arc 135. For example, FIG. 7B illustrates a resilient element having 3 diametric cuts or 6 radial cuts to form 6 flaps. The resulting arc 135 for a resilient element having 6 flaps is larger than the arc resulting from a resilient element of the same size having 4 diametric cuts or 8 radial cuts to form 8 flaps as shown in FIG. 7C. Increasing the number of cuts 104, and therefore the number of flaps 103, reduces the arc 135. In addition, length of the cuts 104 affects the arc 135. Changing the geometry of these flaps, and reducing the arc 135, may enable different materials to be used for the resilient element 101. For example, providing more flaps, reduces the arc and may allow the elastic material to be less elastic, and still rebound to its original shape, or nearly its original shape, after opening the flaps 103. For example, cardboard may be an appropriate material if there are more flaps, and smaller arcs. If there are 4 or 6 flaps, the material may need to be more resilient, such as, for example, silicon, latex, rubber or plastic. In embodiments, the elastic material may be, for example, silicon, latex, rubber, plastic, paper, aluminium foil, paper tape, laminated layered PLA on a paper layer or cardboard. Where more elasticity is required, the material may be selected from silicon, latex, rubber or a combination. In embodiments, the resilient element can be, for example, less than 0.5 mm in thickness.

Further, as shown in FIGS. 7D, 7E and 7F, the length of the cuts may also affect the size and shape of the flaps and the arc 135. The size of the flaps 103, the length of the cuts 104 and the geometry of the arc 135 may be adjusted, in correlation with the material of the resilient element, to optimize the ability of the resilient element to be closed before and after use, and to open to form a suitable airflow pathway during use when the inhaler article 100 is inserted into the holder 200 to form the inhaler system 300. FIGS. 7D, 7E and 7F illustrate pre-cuts 104 which form flaps 103, where the pre-cuts extend about 90% of the diameter of the inhaler article (FIG. 7D), about 78% of the diameter of the inhaler article (FIG. 7E) and about about 65% of the inhaler article (FIG. 7F). FIG. 7G is an illustration of an embodiment of the inhaler article having a central aperture 105 and flaps 103. In embodiments, the resilient element 101 is pre-cut to form at least 4 flaps. In embodiments, the resilient element 101 is pre-cut to form at least 6 flaps. In embodiments, the resilient element 101 is pre-cut to form 4, 6 or 8 flaps. Such number of flaps provides for the arcs areas of the resilient element to provide good flexibility and resistance to tearing ((when pushed into the holder 200), as well as an appropriate rigidity to come back to its initial position when the article is removed from the holder 200. In embodiments, the cuts extend about 65% to 95% of the diameter of the resilient element 101.

In embodiments, the cuts have the same length and cross at the center of the resilient element and have similar angles between them providing symmetrical distribution of a force exerted on the flaps when pushed into the holder 200. Providing this symmetry may assist with preventing tearing of the resilient element upon inserting the inhaler article into the holder and may contribute to the desired opening and closing of the resilient elements.

In embodiments, the cuts 104 of the resilient element 101 are indicia 155. That is, the cuts of the resilient element are symbol indicia 156. In embodiments, the flaps 103 of the resilient element 101 are symbol indicia 156. In embodiments, the resilient element is colored to have color indicia 157. In embodiments, the resilient element 103 has both color indicia 157 and symbol indicia 156. The resilient element as shown in FIG. 7 provides indicia 155.

FIG. 8A, is an illustration of an embodiment of the inhaler article 100 of the present disclosure having a resilient element 101 at the upstream end forming the upstream end face 152, where the resilient element has a central aperture 105. FIG. 8A shows that the resilient element 101 can form the upstream end face 152 of the inhaler article. The resilient element can be colored to provide color indicia 157. The central aperture 105 can provide symbol indicia 156. FIG. 8B is another illustration of inhaler articles 100 of the present disclosure having a resilient element 101 at the upstream end, where multiple inhaler articles 100 can have multiple color indicia 157. That is, different colors may be used for color indicia 157. These colors may indicate different origins, different flavors, different strengths or other information to the user. FIG. 8C is an illustration of an embodiment of the inhaler article 100 of FIG. 5, having indicia 155 at the upstream end face 152 of the upstream element 18. FIG. 8C illustrates that the indicia 155 can be symbol indicia 156. However, color indicia 157 may also be used. In embodiments, the diameter of the central hole may be about 30% of the disc diameter, less than 30%

FIG. 9A, FIG. 9B FIG. 9C and FIG. 9D are illustrations of embodiments of the inhaler article 100 of the present disclosure. FIG. 9A is an illustration of the resilient element 101, having an applied ring of glue 108, prior to affixing the resilient element 101 to the upstream end 120 of the inhaler article 100. FIG. 8B is a photograph of an embodiment of the upstream end 120 of the inhaler article 100 before the resilient element 101 is affixed. FIG. 9C is an illustration of the application of a resilient element 101 to the upstream end 120 of the inhaler article 100. FIG. 9D shows an illustration of the inhaler article 100 after the resilient element has been affixed to the upstream end 120 of the inhaler article 100.

FIG. 10A, FIG. 10B and FIG. 10C illustrate manufacturing equipment 400 and methods for manufacturing embodiments of the inhaler article 100 having a resilient element 101 affixed to the upstream end 120 of the inhaler article 100. This manufacturing equipment 400 operates to cut out round diskettes of resilient material to form resilient elements 101 that fit at the upstream end 120 of the inhaler article 100. FIG. 10A shows a ribbon cutter 403 which has a feeder reel 401, a ribbon 402, a cutter 403, a cutter stage 405 and a take-up reel 404. In use, as shown in FIG. 10A, the feeder reel 401 contains uncut ribbon of resilient material 402. This uncut ribbon of resilient material 402 is presented on a cutter stage 405 to a ribbon cutter 403. The cutter 403 cuts round diskettes of resilient material to create resilient elements 101. In addition, the cutter 403 may make cuts in the resilient elements to form flaps 103, making a resilient element 101 having flaps 103. Or, the cutter 403 may cut a central aperture 105 in the diskette to form resilient elements 101 having a central aperture 105. Alternatively, cuts 104 or central apertures 105 may be made in the resilient elements 101 in a separate cutting step or in an assembly step. After the diskettes are cut from the ribbon 402, the used ribbon 406 proceeds to a take-up reel 404. FIG. 10B shows a glue station 500 having a glue stage to optionally provide a ring of glue to the diskettes or resilient elements in the manufacturing process. FIG. 10C shows an element holder 600 holding three resilient elements which, in use, are presented to the glue stage 502 to apply glue to the cut resilient elements during the manufacturing process.

FIG. 11A and FIG. 11B illustrate manufacturing equipment and methods for manufacturing embodiments of the inhaler article 100 having a resilient element 101 affixed to the upstream end 120 of the inhaler article 100. FIG. 11A shows the cutting step wherein a ribbon 402 of resilient material is presented to a cutting stage 405, and the ribbon cutter 403 addresses the cutting stage 405 with the ribbon 402 of resilient material. The ribbon cutter 403 cuts diskettes of resilient material from a ribbon 402 of resilient material. The cutting may occur by press-cutting, knife-cutting, laser cutting, or by any means. FIG. 11B shows step of removing the diskettes of resilient material from the cutting stage 405. Once the cut has been made, the diskettes of resilient material may be removed from the cutting stage 405 to proceed to the next step in the manufacturing process. In addition, the cut ribbon 406 proceeds to the take-up reel and fresh, un-cut ribbon will be presented to the cutter stage 405 so that the cutting step can be repeated. The cutting step may also include cutting lines in the resilient element to form flaps, or cutting a central aperture in the resilient element, or cutting a combination of a central aperture and flaps in the resilient element. The cutting step may also include cutting indicia 155 into the resilient element 101. The cutting step may include cutting lines, flaps or a central aperture into the resilient element. Cut lines 104, flaps 103 or a central aperture 105 may be indicia 155.

FIG. 12A, FIG. 12B, and FIG. 12C illustrate manufacturing equipment and methods for manufacturing embodiments of the inhaler article 100 having a resilient element 101 affixed to the upstream end 120 of the inhaler article 100. FIG. 12A, FIG. 12B and FIG. 12B illustrate the gluing step of the manufacturing process. FIG. 12A illustrates a step of presenting the resilient element to the gluing station. FIG. 12B illustrates the step of loading a glue ring 502. Glue may be extruded onto the stage from a reservoir of glue located under the glue stage. Alternatively, glue may be presented in dots, a discontinuous ring, a thick ring, a thin ring or any other shape to provide glue to the glue station. FIG. 12C illustrates the step of applying glue to the resilient element. FIG. 12A illustrates the step of presenting the cut resilient element 101 to the glue station 500. The glue stage 501 has a glue ring 502. The resilient element 101 is presented to the glue stage 501 on an element holder 600. FIG. 12A shows a glue ring 502 before glue has been provided to the glue ring 502. FIG. 12B illustrates the glue stage 501 and the glue ring 502 after glue has been provided to the glue ring 502. In other words, the glue ring has been loaded with glue, as shown in FIG. 12B. In embodiments, glue may be presented to the glue ring 502 by pressing the glue into the glue ring 502 from a reservoir of glue below the glue stage 501. FIG. 12C illustrates the step of pressing the cut resilient element 101 onto the glue stage 501 to provide glue to the resilient element 101. Appropriate glues may include starch adhesives such as dextrin, casein-based adhesive, polyamide blue, hot melt glue, cyanoacrylate, organic glues, or any other suitable glue.

FIG. 13A, FIG. 13B, and FIG. 13C illustrate manufacturing equipment and methods for manufacturing embodiments of the inhaler article having a resilient element 101 affixed to the upstream end 120 of the inhaler article 100. FIG. 13A illustrates a perspective view of the resilient element 101 having an applied ring of glue 503, after the manufacturing step of FIG. 12C. FIG. 13B illustrates the presentation of the inhaler article 100 to the glue-side of the resilient element 101 having an applied ring of glue 503. FIG. 13C illustrates the inhaler article having a resilient element 101 affixed to the upstream end 120 of the inhaler article 100. In embodiments, the resilient element 101 is an upstream element. In embodiments, the resilient element 101 comprises indicia 155. In embodiments, the indicia are color indicia 157. In embodiments, the indicia are symbol indicia 156. In embodiments, the resilient element 101 comprises both color and symbol indicia.

For the purpose of the present description and of the appended claims, except where otherwise indicated, all numbers expressing amounts, quantities, percentages, and so forth, are to be understood as being modified in all instances by the term “about”. Also, all ranges include the maximum and minimum points disclosed and include any intermediate ranges therein, which may or may not be specifically enumerated herein. In this context, therefore, a number A is understood as A±10% of A. Within this context, a number A may be considered to include numerical values that are within general standard error for the measurement of the property that the number A modifies. The number A, in some instances as used in the appended claims, may deviate by the percentages enumerated above provided that the amount by which A deviates does not materially affect the basic and novel characteristic(s) of the claimed invention. Also, all ranges include the maximum and minimum points disclosed and include any intermediate ranges therein, which may or may not be specifically enumerated herein.

1. An inhaler article for insertion into a holder to form an inhaler system for providing a compound to lungs of a user, the inhaler article comprising:
a tubular body extending along a longitudinal axis from a downstream mouthpiece end to an upstream end;
the upstream end comprising an upstream element;
the upstream element comprising an upstream end surface; and
wherein the upstream end surface comprises colored indicia being a different color than the upstream end surface the colored indicia is disposed on.

2. The inhaler article of claim 1 wherein the colored indicia comprises at least one symbol.

3. The inhaler article of claim 1 wherein the tubular body comprises, in linear sequential arrangement, from the upstream end to the downstream mouthpiece end:
the upstream element;
a capsule cavity containing a capsule;
the capsule containing dry powder active ingredient; and,
a blocker element.

4. The inhaler article of claim 3 wherein the upstream element comprises a resilient element.

5. The inhaler article of claim 4 wherein the resilient element is affixed to the upstream end of the tubular body.

6. The inhaler article of claim 4, wherein the resilient element comprises cuts which form flaps.

7. The inhaler article of claim 4, wherein the resilient element comprises a central aperture.

8. The inhaler article of claim 4, wherein the resilient element comprises silicon, latex, rubber, or a combination thereof.

9. The inhaler article of claim 3, wherein the blocker element comprises a filter.

10. The inhaler article according to claim 3 wherein the tubular body comprises, in linear sequential arrangement, from the upstream end to the downstream mouthpiece end:
the upstream element,
a rod of aerosol-generating substrate located downstream of the upstream element,
an aerosol-cooling element located downstream of the rod of aerosol-generating substrate,
a blocker element located downstream of the aerosol-cooling element, and
an outer wrapper circumscribing the upstream element, the aerosol-cooling element and the blocker element.

11. An inhaler article according to claim 4, wherein the resilient element opens to an open position in response to an opening force, and upon removal of the opening force, closes to a closed position;
wherein when the resilient element is in the closed position, the resilient element is sufficiently closed to retain the capsule in the capsule cavity between the downstream mouthpiece end and the resilient element.

12. An inhaler system comprising:
a holder, wherein the holder comprises a piercing element; and,
an inhaler article according to claim 1 inserted into the holder.

13. The inhaler system according to claim 12, wherein the holder provides an opening force to open the upstream element.

14. The inhaler system according to claim 12, wherein the holder comprises a heater.

15. A pack containing a plurality of inhaler articles according to claim 1, and wherein at least one upstream end surface comprising colored indicia of at least one inhaler article contained in the pack is visible when the pack is open.
